(19) United States
(12) Patent Application Publication
(10) Pub. No.: US 2008/0310995 A1
(43) Pub. Date: Dec. 18, 2008

(54) Method, Device and System for Thermal Processing

(76) Inventors: Stanley E. Charm, Boston, MA (US); James A. Farago, Salisbury, MA (US); James R. Bowden, North Scituate, RI (US)

Correspondence Address: Charm Sciences, Inc.;Richard J. Long, 659 Andover Street, Lawrence, MA, 01843, US

(21) Appl. No.: 10/582,457

(22) PCT Filed: Dec. 10, 2004
(86) PCT No.: PCT/US04/41278

§ 371 Date: Jun. 12, 2006

Related U.S. Application Data
(60) Provisional application No. 60/529,341, filed on Dec. 12, 2003.
(60) Provisional application No. 60/609,935, filed on Sep. 15, 2004.

(51) Int. Cl. A61L 2/12; A61L 2/04

(52) U.S. Cl. 422/21; 422/1; 422/186

(57) ABSTRACT

The system and method features continuous, high-temperature, short-time thermal processing of biological materials, including protein, that is suitable for large scale production at rapid flow rates. The thermal processing can include rapid temperature increases and rapid cooling. The thermal processing can be used to reduce or eliminate contaminants, such as viruses and bacteria, from biological materials such as protein solutions.

### REFERENCE TO PRIOR APPLICATIONS

This application is based on and claims priority from U.S. Provisional Patent Application Ser. No. 60/529,341, filed Dec. 12, 2003, and U.S. Provisional Patent Application Ser. No. 60/609,935, filed Sep. 15, 2004, both of which are incorporated by reference.

### BACKGROUND

It is desirable to sterilize, pasteurize or otherwise heat treat heat-sensitive material such as biological fluids for example blood plasma, serum, milk, immunoglobulins, tissue culture, or tissue-type or other material, by heating such heat-sensitive material to high temperatures for very short time periods without diminishing desirable properties of the material. U.S. Pat. No. 4,839,142, issued Jun. 13, 1989, and U.S. Pat. No. 4,975,246, issued Dec. 4, 1990, describe high temperature, short time heating of biological materials to destroy undesirable microorganisms, pathogens, and/or viruses in a sample without substantially degrading the material itself. The heat-sensitive material is heated rapidly, such as by microwave energy, to a selected temperature, held for a very short holding time, and then cooled and recovered. Typically, the material is heated to a pre-selected temperature at which the rate of reduction or destruction of undesirable micro-organisms is greater than the rate of destruction of the heat-sensitive material itself. After inactivation, the material is cooled to stabilize the material.

One area in which the use of continuous, high-temperature, short-time heat treatment of biological materials, such as proteins, can be important is in the development of methods for producing biological products such as biologics-based pharmaceuticals, vaccines and the like. Existing systems have been used in small-scale manufacturing and during the development stages, where sample sizes are relatively small compared to the batch sizes expected in large scale production processing. One existing system, the UltraTherm® (system (Ultra Therm is a registered trademark of Charm Bioengineering, Inc., Lawrence Mass.), is equipped with a 5 kilowatt (kW) power supply and 2450 megahertz (MHz) microwave generator (also known as a magnetron). The 5 kW UltraTherm® (is capable of processing about 60 liters per hour (L/hr). It is conventional to process complete batches within a single work-day (typically 5-8 hours), so the flow rate of existing systems limits the usual batch size to about 300 liters per day, far below the batch sizes desired for certain production-scale manufacturing. (See also U.S. Pat. No. 5,389,335, issued Feb. 14, 1995, hereby incorporated by reference.)

It is, therefore, desirable to provide a system for the continuous, high-temperature, short-time heat treatment of biological materials that is suitable for large scale production at rapid flow rates. It is further desirable to provide a system for continuous, high-temperature, short-time heat treatment of biological materials that has design features optimized for modern production facilities. It is further desirable to provide a system with flexibility to maximize the possible time/temperature profiles achievable at a variety of flow rates.

### SUMMARY

Disclosed is a system and method for thermal processing of a material such as a heat-sensitive fluid material. In one aspect, the system utilizes a source of microwave energy for heating. The microwave energy source can be configured to generate a frequency of, for example, greater than about 850 MHz, preferably greater than about 1000 MHz, most preferably 2450+/−50 MHz, and has a power capacity of at least 10 kW, in the range of about 10 to about 100 kW, for example 20 kW, 30 kW, or 40 kW. The system can be designed to accommodate a flow stream with a flow rate of up to and above 80 liters per hour, for example above about 300 liters/hour. The flow stream can flow through a waveguide within tubing secured to a removable plate.

Also disclosed is a method of reducing, inactivating or destroying an agent, such as a micro-organism, such as a bacteria, or pathogenic agent, such as a virus, in a fluid sample of a heat-sensitive material. The heat-sensitive material can include a variety of materials or combinations of materials such as biological materials and including solutions of protein, such as antibody solutions, solutions of protein hydrolysate and solutions of bacteria. The method can include selectively reducing, inactivating or destroying the agent. By selectively reducing or inactivating the agent the desirable properties of the material, such as protein activity or nutrient value, can be maintained while the agent is reduced, inactivated or destroyed. The method may include: a) providing a heat-sensitive product in a fluid flow stream having a flow rate of greater than about 80 liters per hour; b) exposing the product flow stream to microwave energy of greater than about 1000 MHz for a pre-selected time, for example less than about 0.5 seconds, sufficient to raise the temperature of the product in the flow stream to a pre-selected temperature, for example in the range of about 60 degrees C. to about 130 degrees C., so as to reduce, for example by one or more logs, or inactivate the agent without substantially altering the desirable properties of the product, such as protein activity; and c) rapidly cooling the product flow stream. In an embodiment, the method includes utilizing a frequency of microwave energy within the S band spectrum, for example about 2450+/−50 MHz or the L band, for example about 915 MHz.

An embodiment includes a 30 kW, or more than about 30 kW, microwave system for high volume processing at temperatures and times that inactivate or destroy viruses and/or bacteria, but can retain some, part or all of the favorable properties, such as protein activity, of the material being processed. Such a system can also be operated at times and temperatures sufficient to sterilize or pasteurize biological fluids. The acceptable maximum time temperature profile may depend at least in part on the stability of the biological product being processed, the buffers used, the amount of heat required to inactivate, reduce or destroy the target contaminant and, in the case of protein, the amount of remaining activity required for the application to be practical. For example, some applications may require upwards of 90% protein activity to remain while other may require as little as 10% remaining protein activity for the application to be practical.

The rates of destruction of viruses compared to proteins generally favor heating biological fluid to maximum temperature for the shortest period of time possible. Systems taking advantage of this phenomenon are described in U.S. Pat. Nos. 4,839,142, 4,975,246, 5,389,335, and 5,539,673, each of which is hereby incorporated by this reference. See, also, Charm et al. High-Temperature Short-Time Heat Inactivation of HIV and Other Viruses in Human Blood Plasma, Vox Sang 1992; 62:12-20. An aspect of the herein described system and method is the flexibility to adjust flow rates, heating rates and cooling rates to accommodate a wide range of products and contaminants depending on the characteristics of the product and contaminants. For example, the Arrhenius constants of the particular products and contaminants can be used to predict both whether the system will be useful for a particular product/contaminant combination and the system parameters, that may be required to achieve target log reduction of contaminant and preservation of product.

The method and system can be useful for achieving multilog reduction of agents, such as pathogenic agents, such as viruses in a manufacturing environment requiring large volume processing, and, therefore, large flow rates of biological fluids. In an example, the method and system includes a 30 kW microwave power supply to a microwave generator and flow rates of about 300 to about 450 liters per hour. Such a 30 kW system provides ultra rapid heating and cooling to maintain the desirable properties of the material, such as protein activity, while destroying viruses or other contaminants.

Another aspect is a system for processing biological products at high flow rates. To heat fluid evenly throughout the system, it is preferable to maintain turbulent flow. To maintain turbulent flow, the internal diameter (ID) of the various portions of the flow path exposed to heating and cooling conditions are preferably minimized to maintain an adequately high Reynolds number. However, small ID tubing can result in high system pressure and pressure drops. At high pressures, depending on specifications of the various portions of the flow path, there is a risk of fluid pathway rupture. As a result, while minimizing the ID of the various flow path sections, system pressure changes should also be minimized.

It is another aspect to provide a heating system in which system pressure is optimized by directing increased flow rates and, therefore, pressure changes to the portions of the flow path in which the heat sensitive material being processed is most vulnerable to denature and where maximum rapid heat-transfer is desired. For example, utilizing tubing within system cooling sections generally with a smaller ID than in heating sections.

Another aspect is the modular design of the system. In the modular system various modules of the complete system are maintained remotely. For example, the fluid processing unit, the utility unit, the power supply unit, the central processing unit, and the supervisory controls unit can all be maintained separately and in remote locations, for example a separate room. Alternatively, all such units can be maintained together. Use of modular components confers significant advantages when using the system in a production suite having limited floor space. In such cases, certain modules, such as one or both of the power supply module and the utility module, can be moved to remote locations while remaining functionally, electrically or otherwise integrated to other portions of the system.

Another aspect is a cost efficient method for removing and replacing the portion of the flow path exposed to microwave heating. If the microwave section of the flow path clogs or is contaminated it needs to be replaced. Costly flow paths, or flow paths that cannot be easily removed, are undesirable. It is, therefore, useful to provide an inexpensive and easily removable flow path cartridge. The cartridge plate can be constructed of, for example, of aluminum or stainless steel with tubing attached to and extending downward from the plate. Useful tubing includes microwave transparent tubing such as, for example, TEFLON (TEFLON is a registered trademark of E.I. Du Pont De Nemours and Company Corporation, Wilmington, Del.) tubing. The tubing can be wound in the desired orientation through microwave transparent plastic.

Another aspect is a 30 kW driven microwave system that can approximate the heating and cooling conditions of a smaller system, such as a 5 kW driven microwave system. Such a system will allow easy scale-up of successful research and development, that may utilize the smaller, for example a 5 kW system, to high flow rate processing.

Another aspect is a microwave heating system that includes ultra-sensitive sensors to detect pressure within a waveguide that emit signals in the case of potential leaks. Such a pressure sensing system can include an air or inert gas inlet. Small leaks will cause pressure loss and system shut down.

Another aspect is a heating and cooling system and method for processing heat-sensitive materials in which rapid heating and rapid cooling, of over about 100 degree C. per second, is achieved in a system and method that allows flow rates of over about 80 liters per hour, for example about 300 to about 450 liters per hour. In an embodiment, multiple cooling methods are used. For example, multiple heat exchange coolers with at least one able to cool at a rate of above about 100 degrees per second. Multiple coolers can be used to allow system flexibility so that target time/temperature profiles can be achieved.

In another aspect, one of the multiple coolers can be a tube and shell heat exchanger with or without a secondary cooling jacket. The tube and shell heat exchanger can be positioned to provide timely cooling, as heated fluid leaves the microwave applicator, and can be connected to a secondary cooler, such as a plate heat exchanger. The tube and shell heat exchanger can also be used as the sole cooling method.

Another aspect is a multi-clamp system that can be used to prevent microwave leakage. In an embodiment, the multi-clamp system is used for tightly sealing a cover plate over a waveguide. The multi-clamp system can be used to help control microwave leakage. The clamps can be attached to a single bar characterized in that the clamping an unclamping of the multiple clamps is leveraged using the single bar. Such a multi-clamp system can be used in conjunction with a highly sensitive pressure change sensor and proper system sealing and grounding to prevent microwave leakage from the waveguide or applicator. The pressure change sensor can include an air impermeable pressure window at the top section of a waveguide below the cover to the waveguide which cover sits on top of an applicator. Within the applicator can be included a conductive gasket and a quarter wave choke.

### BRIEF DESCRIPTION OF THE DRAWINGS

FIG. 1 is an overview of an embodiment of the system.

FIG. 2 is a perspective view of a fluid processing unit also shown in FIG. 3.

FIG. 3 is a front view of a fluid processing unit.

FIG. 4 is a partial rear view of the back side of the fluid processing unit shown in FIGS. 2 and 3.

FIG. 5 is a perspective view of a utility skid for providing cooling and heating fluids to a fluid processing unit such as the unit shown in FIGS. 2, 3 and 4.

FIG. 6 is a schematic drawing of a waveguide connected to a magnetron box.

FIG. 7 is a perspective view of a microwave applicator with T-fittings and cover.

FIG. 8 is a perspective view of a microwave applicator with microwave cover removed showing opening into waveguide.

FIG. 9 is a schematic side view of a microwave cover with connected tubing that can be inserted into the opening in a waveguide.

FIG. 10 is a schematic side view of a microwave cover with an alternative method of winding the connected tubing that can be inserted into the opening in waveguide.

FIG. 11A is a perspective view showing a microwave cover multi-clamp locking mechanism. FIG. 11B is a top view of a microwave cover locking mechanism. FIG. 11C is a side view of a microwave cover locking mechanism.

FIG. 12 is a perspective view of a microwave cover connected to a heat exchanger and showing microwave transparent tubing holders.

FIG. 13 is a cross-sectional drawing view of a non-jacketed intercooler positioned between the outlet of the microwave and a funnel plug connected to the inlet of a cooling plate heat exchanger.

FIG. 14 is an enlarged cross-section of the non-jacketed intercooler shown in FIG. 13.

FIG. 15 is a front perspective view of a funnel plug with tapered end not shown.

FIG. 16 is a rear perspective drawing of a funnel plug with tapered end shown.

FIG. 17 is a cross-sectional drawing of the funnel plug shown in FIGS. 15 and 16.

FIG. 18 is a cross-sectional drawing of a jacketed intercooler showing product and coolant flow paths.

FIG. 18A is a partial cross-sectional drawing of a jacketed intercooler showing product and coolant flow paths with arrows showing coolant flow.

FIG. 19 is a plan view showing placement of a jacketed intercooler shown in FIGS. 18 and 18A and showing the direct connection to a secondary cooler (in this figure the secondary cooler is a cooling plate heat exchanger).

FIG. 20 is a graph of cooling rates from various peak temperatures at a flow rate of 425 liters per hour using a jacketed intercooler.

FIG. 21 is a perspective of a flow path of a fluid processing unit such as depicted in FIGS. 2 and 3 showing fluid inlet and outlets.

FIGS. 22 and 23 are graphs showing results from experiments to test the activity of beta galactosidase and glucose oxidase after heating to a range of temperatures and cooling with a non-jacketed intercooler.

FIG. 24 is a graph showing results from experiments to test the activity of glucose oxidase after heating to a range of temperatures and cooling with a jacketed intercooler connecting directly to a secondary cooler.

FIG. 25 is a graph comparing log reduction of E. coli to reduction of glucose oxidase activity in a 5 kW system compared to a 30 kW system using a jacketed intercooler.

FIG. 26 is a graph of a time-temperature curve comparing a 5 kW system to a 30 kW system using a jacketed intercooler.

FIGS. 27A and 27B are schematic drawings of system fluid flow.

### DEFINITIONS

By fluid processing unit we mean the portion of the system in which peak heating and rapid cooling of product occurs. Product includes material to be thermally processed or otherwise passed through the flow path of the system.

Funnel plug means a reducing fitting or adapter designed to be positioned between tubing of varying diameters and to provide a smooth transition to minimize system pressure changes.

By power supply unit we mean the power supply providing energy to the heater, for example the microwave generator (magnetron).

By supervisory control unit we mean the hardware and software containing the operator interface.

Auto-tuner means the mechanism usually attached to a waveguide and projecting within a waveguide that adjusts the microwave field.

Dummy load means the section of a waveguide that receives and absorbs energy not absorbed by the load.

Waveguide means a microwave chamber through which microwave energy is propagated from a magnetron to the material to be heated.

Applicator means the end of a waveguide on top of which there may be a cover. The tubing exposed to the microwave can extend into the waveguide.

Magnetron means microwave generator.

The cartridge includes the portion of the flow path that will be exposed to microwave energy.

By intercooler we mean a cooler that bridges a gap, for example between a heater and another cooler, to provide immediate cooling. By jacketed intercooler we mean an intercooler with a fluid path surrounded by two chambers of coolant, with one chamber jacketing the other. By non-jacketed we mean a single chamber of coolant surrounding a flow path.

### DETAILED DESCRIPTION

In an embodiment, the system is an entirely self-contained, GMP (good manufacturing practices) compliant, continuous flow, high temperature, short time heating system with complete computer controls and safety interlocks. Pumps, heat exchangers, valves, tubing and instrumentation can be built on mobile stainless steel frames with wheels. Stainless steel and disposable components ease maintenance. The system can be PLC (programmable logic controller) controlled and can be operated by the user through an advanced touch screen display featuring hierarchical organized screens which provide all primary control parameters. System software can protect against improper operation. The process screen can give a flow chart view of the system with most major parameters displayed in real-time.

In an embodiment, microwave energy is used as the heat source. The microwaves can be produced by a magnetron tube and fed through a waveguide to a microwave applicator. Specially configured tubing, for example of TEFLON or PEEK (polyetheretherketone), can be positioned inside the microwave field to achieve maximum energy transfer to the product and uniform heating and, thus, the highest temperature in the shortest time. Hold times at the peak temperature may be varied by adjusting the flow rate and/or the internal diameter of the tubing. The peak, or approximate peak, fluid temperature is monitored by either or both a non-intrusive infrared temperature sensor and RTD, and is controlled by adjusting the output power of the magnetron.

In an embodiment, product is drawn through a pump, such as an autoclavable, motorized, variable speed, 2.6 ml/revolution, positive displacement pump, into a first heat exchanger where it is preheated. Product then passes through the microwave field where the temperature is rapidly increased. The product then flows into one or more cooling sections including, for example, cooling heat exchangers, where it is rapidly cooled, thereby halting or slowing product destruction.

Precisely controlled valves allow switching between product and a flushing solution to accurately control the recovery of the thermally processed product.

The system is designed to meet the throughput requirements of today's high volume production facilities in a throughput capacity of, for example, 360 liters per hour. The system is highly automated and user friendly. The system can be modularized to maintain a small footprint in the expensive production suite. System components, including the microwave power supply, utilities modules and control module, can be remotely located. Remote modules can be networked and computer controlled. Using stainless steel, such as in the microwave module, with hinged doors all around for accessibility, the system can be designed for easy maintenance and cleaning. The waveguide section can be interlocked with redundant safeties. Features of the system can include about 300 to about 450 L/H continuous throughput; ultra short time heating; viral log reduction capabilities; minimal product loss; accurate and repeatable temperature control; 2450 MHz microwave technology; industrial grade components; no chemicals to remove; no clogged filters to replace; small footprint in the manufacturing suite; and modular movable system.

An embodiment is a modular system for heat treatment of biological fluids at increased flow rates. A modular design allows the fluid processing unit to be separated from other portions of the system. By such a separation, the fluid processing unit can be isolated, for example in a temperature, humidity or other controlled environment room. The fluid processing unit can contain all product contact points. As a result, the strict sanitation requirements relative to components that contact pharmaceuticals products and raw materials need not be applied to remote units, thereby saving time and expense.

The modular design also can allow for sharing of costly components. For example, in embodiments utilizing a microwave heating system, one power supply unit can be alternated between two or more fluid processing units. Alternating the power supply between units can decrease down time during switch over from one product to another or during cleaning. In addition, sharing of fluid processing units allows for a more cost effective approach to avoiding cross-contamination between products. For example, one fluid processing unit can be assigned to one particular product while a utility unit or power supply unit can be shared.

The modular design of the system can be even more critical when large magnetrons (microwave generators) are used. For example, magnetrons that generate 895 or 915 MHz frequency microwaves can currently utilize somewhat larger power supplies, up to 100 kW, whereas magnetrons that generate 2450 kW frequency microwaves have power capacity in the range of about 1 kW to about 30 kW. Larger power supplies may require more physical space.

In an embodiment the modular system includes three modules: the power supply unit, the fluid processing unit (including system controls) and the utility unit. In another embodiment the modular system includes four modules (or units): the power supply unit, the fluid processing unit, the utility unit and a control unit.

### Power Supply Unit

In one embodiment, the power supply unit includes four identical 12 kW inverter modules, creating a 48 kW power supply unit. As such, the power supply is itself modular; each 12 kW inverter module can operate independently and one inverter unit at a time can be replaced. In this particular embodiment, the power supply includes four identical but separate switching power supplies with series additive outputs. Each module converts 480 volts alternating current (480 vac) into direct current at 16 kVdc and 2.45 amp. It will be appreciated that although a 48 kW power supply is used with a 30 kW magnetron, a smaller power supply is sufficient for a lower kW magnetron. For example, three or even two 12 kW inverter modules could be used to operate a 20 kW magnetron. Similarly, a larger power supply would be required for a larger magnetron. Alternatively the power supply unit can include a single inverter module as the full power source.

Whether the power supply includes a single inverter module or several linked together, it can be provided in a housing that is separate from the fluid processing unit. The power supply can be provided on a skid with wheels for easy movement. In an embodiment, a single power supply or sub-modules of a single power supply can be used to operate multiple fluid processing units. In that way, the cost of purchasing several systems can be reduced, the flexibility in the size of the space allocated to the fluid processing unit is increased and the cleaning requirements for the power supply will not be as stringent as that for the fluid processing unit.

In one modular embodiment, the power supply receives instructions from a control module or supervisory control unit over a network, for example, a Profibus network or wireless network and supplies power through a high voltage cable to the magnetron over a filament and magnet control cable.

### Utility Unit

Adding to the modularity of the system, the utility unit, in one example including both coolant fluid sources and optional heating fluid sources, can be provided separately from the other system modules. For example, the utility unit can be remote from the fluid processing unit, such as in a separate room or the same room apart from the fluid processing unit. In addition, as with the power supply unit, one utility unit can be used to operate separate fluid processing units.

In a modular embodiment, the utility unit receives instructions from the control module or supervisory control unit over a network, such as a Profibus network through a cable or through a wireless connection.

In an embodiment the utility unit is on wheels as a unitary, movable unit. The utility unit can include two parallel sides: the heating side and the cooling side. Each side can be a self-contained closed loop system with cooling capacity supplied by an independent cooling source, for example cold water/glycol or cold water flowing through heat exchangers. In an embodiment, multiple closed loop coolant or heating systems can be included. In a particular embodiment described herein, there are two coolant loops and one heating loop.

### Cooling Loops

The cooling portion of the utility unit can include one or multiple coolant supply reservoirs. In a particular embodiment, two coolant reservoirs are provided: one to supply the heat exchanger(s) that cool product after heating (the primary coolant loop) and one to supply cooling fluid to components of the system requiring cooling (secondary coolant loop). The fluid within the cooling reservoirs is maintained at low temperatures by circulating through a heat exchanger utilizing plant supplied coolant.

In an embodiment, the primary coolant loop includes a coolant mixture, for example, a coolant liquid that is a mixture of 90% deionized water to 10% glycol. Various combinations can be used for coolant including 100% cold water. The primary coolant loop is a closed loop system including a coolant liquid reservoir. In an embodiment for processing large volumes, for example in a 30 kW system, the reservoir capacity can be approximately 400 cubic inches. Reservoir size can easily be adapted to changes in system requirements. In an example, liquid flows from the reservoir to the suction side of a motorized pump. From the pump, fluid flows through a heat exchanger, to cool the fluid exiting the reservoir. The heat exchanger can utilize coolant such as chilled water from an external source. The fluid then flows from the utility unit to the fluid processing unit. In an example, the fluid processing unit utilizes the primary coolant loop fluid to cool heated material leaving the applicator/waveguide of the fluid processing unit. The cooling of the heated material can be accomplished using one or multiple heat exchangers adapted to the particular requirements of the system. In another embodiment, the primary coolant loop also provides coolant to an optional intercooler, such as a tube and shell, positioned between the outlet of the applicator/waveguide and the inlet to the cooling plate heat exchanger.

After flowing through the cooling plate heat exchangers of the fluid processing unit the primary coolant loop fluid returns to the utility unit. Upon return from the fluid processing unit the fluid can flow through a flow meter, to verify proper pump functioning prior to flowing back to the reservoir. Within the reservoir two level switches can be provided to monitor both maximum and minimum desired fluid levels.

In an embodiment, a secondary coolant loop includes, for example, 100% deionized water. Such a secondary coolant loop can be used to provide coolant for a variety of purposes including for either multiple parallel cooling circuits or, alternatively, a continuous flow series loop or a combination thereof. The advantage of parallel cooling circuits is that fluid temperature to each parallel circuit is similar as compared to circuits in series in which coolant first flows from one cooling circuit to the next, absorbing heat but not substantially dissipating heat. In both the parallel and series cooling circuit design, coolant flows to each cooling circuit separately and then is circulated back to the reservoir.

The secondary cooling loop can begin from a reservoir, proceed to the suction side of a secondary coolant loop pump, such as a pump identical to that used for the primary cooling loop, and then leave the utility unit and enter the fluid processing unit.

Within the fluid processing unit, in a multiple parallel cooling circuit embodiment, the secondary coolant loop can include various circuits, such as: 1) magnetron tube cooling circuit; 2) applicator cooling circuit; 3) auto-tuner cooling circuit; 4) cabinet air cooling circuit; 5) dummy load cooling circuit. It is possible also to include a circuit to the optional intercooler (for example a shell and tube heat exchanger or jacketed shell and tube heat exchanger) positioned between the outlet of the applicator and the inlet to the cooling plate heat exchanger. Within each cooling circuit can be multiple branch cooling circuits. Also within particular loops, such as the magnetron loop, can be filters to prevent particles from clogging small orifices. In an example of a microwave system including an auto-tuner, the auto-tuner cooling circuit itself includes multiple branch cooling circuits, for example, above and below the auto-tuner. In another example, a cabinet air-cooling circuit includes fans to blow cool air off a cooling circuit and into the cabinet.

All coolant loops can have temperature sensors to verify the temperature of the coolant. For example, a resistance temperature device (RTD) can be used to measure temperature of the coolant fluid. In one embodiment, an RTD is situated prior to the entry of the secondary coolant loop fluid to the magnetron tube cooling circuit. Efficient placement of RTD's in general can be directed to the most vulnerable locations requiring coolant, for example the locations generating the most heat such as the magnetron tube. RTD's can also be located in what are known in the art as thermowells. Placement within thermowells allows movement of RTD's from one location to the next without disrupting coolant flow. Fittings, such as SWAGELOCK (Swagelock is a registered trademark of Swagelock Company, Solon, Ohio) fittings, can be used to secure RTD's into the various thermowells.

Multiple flow switches can be included through the cooling and heating flow to verify proper system performance. For example, flow switches can be included around the magnetron tube cooling circuit, the auto-tuner cooling circuit and the dummy load cooling circuit. To avoid damage to the equipment and/or injury to personnel, lack of adequate coolant flow through a cooling circuit can automatically shut the system down.

A third or more cooling loops can be provided if necessary, particularly if one or more areas require fluid at different temperatures than other areas. In an embodiment, a third cooling loop provides coolant to an air conditioning unit within the microwave control and power supply.

### Pre-Heating Loop

In an embodiment a pre-heating loop is provided. The pre-heating loop can provide hot water to a preheat plate exchanger of the fluid processing unit. In an embodiment, the pre-heating fluid is stored in a reservoir with capacity of approximately 400 cubic inches. Fluid leaves the reservoir and enters the suction side of a pump where it is pumped to a heater, such as an electric resistance heater or gas heater, or a heat exchanger. Heated fluid flows from the heater by an RTD that checks the temperature of the heated fluid. Heated fluid flows off the utility unit and into the preheat plate exchangers of the fluid processing unit. After employment in the plate heat exchangers, fluid flows through a flow meter and back to the reservoir.

The pre-heating loop can provide the initial heating, within the fluid processing unit, prior to microwave heating. Such a pre-heating loop is optional; all heating can occur within the microwave portion of the system. Providing a heat exchange pre-heating loop can increase the efficiency of the system and help maximize the peak temperature achievable at a given flow rate with a given power supply.

The critical portion of the heating within the fluid processing unit, in the case of proteins, begins at the temperature above which protein denature, of the particular protein being processed, occurs. Above the temperature at which denature occurs, rapid heating is desired so that contaminant destruction can occur quickly followed by rapid cooling. Below the temperature at which product denature occurs, a longer heating time may be acceptable. Thus, utilizing a plate heat exchange element, fed by a heated fluid loop, allows microwave power to be efficiently used for maximum rapid heating above the critical temperature. For example, it may be most efficient to utilize the pre-heat heat exchanger to bring the fluid temperature to just below the temperature of significant protein denature. After reaching that temperature, rapid heating in the microwave can be used to avoid denature, partially or wholly. Parameters chosen will depend on the particular product, such as protein solution, or stabilization buffers used and the particular target virus or bacteria to be destroyed.

### Fluid Processing Unit

In an embodiment, the fluid processing unit includes stainless steel, for example 316 stainless steel, tubing through which product flows. Various size tri-clamp fittings, sanitary valves and sanitary adapters can be used to provide connections within the generally stainless steel flow path. The product flow path can also include TEFLON or other flexible material such as silicon or latex. The choice of material will partially depend on requirements such as whether or not it is necessary that the flow path be autoclavable, the maximum pressure requirements of the system and maximum pressure specifications of the various materials.

The fluid processing unit can have one or multiple processing inlets that can be controlled by valves. In an embodiment including three inlets, at least one inlet is for product. Another inlet can be for saline or other buffers. A third inlet can be used for another buffer, alternative product or to flush the system, for example with sodium hydroxide or other cleaners compatible with the particular flow path material. All inlet sources can be maintained remote from the fluid processing unit, adjacent to the fluid processing unit or attached to the fluid processing unit. Solenoid valves can be used to control the source of product. Product flow for processing is from one or the other inlet through a solenoid or other type of valve into the suction side of a pump that can be controlled by a variable speed drive.

In an embodiment, product flows out from a pump, such as a motorized positive displacement pump, into a parameter check section, including a variety of sensors, meters and other measurement devices, that can be in a variety of possible configurations and orders, including, for example: a bubble sensor using ultra sound to check for air pockets or loss of fluid; a magnetic flow meter, such as a ⅜ inch ID magnetic flow meter connected to a signal converter; a pulsation damper; a pressure sensor; and a ti-clamp flange connection. The pressure sensor can be used to measure the pressure in the flow path of the system immediately prior to fluid entering the pre-heat loop. Undesirable pressure measurements at this point in the system can trigger the system to shut down. Product can next pass an RTD to measure the temperature of the fluid prior to fluid entering the processing section. System variables can be automatically or manually adjusted to accommodate varying temperatures of product entering the system.

In an embodiment, the processing section of the fluid processing unit includes: preheat unit; microwave applicator unit; primary coolant unit; secondary coolant unit. The preheat unit can be used to maximize heating efficiency of the microwaves. Similarly, it is desirable to maximize cooling efficiency and, therefore, a variety of cooling methods and systems, including combinations of cooling methods and systems, may be useful.

In an embodiment, the preheat section includes a flat-plate heat exchanger. In another embodiment, the preheat section includes a tube in shell heat exchanger or other heat exchangers known in the art. In either embodiment, heated fluid can be provided from the pre-heating loop of the utilities unit.

In an alternative embodiment, no pre-heat section, and, therefore, no utility unit pre-heat loop is required. In such an embodiment, all heating is accomplished by a microwave source. Depending on the peak temperature and speed to peak temperature requirements, such a non-preheat system may or may not be practical.

After product exits the pre-heat unit, product temperature can be measured by an RTD. An RTD can provide a signal to a programmable logic controller. The signal can be used to adjust pre-heat temperature by controlling the speed of the hot water pump. If the pre-heat is not providing sufficient heating of the product, the pump speed of the utility unit hot water pump can be increased. If the pre-heat is overheating the product, the pump speed can be decreased. Alternatively, the temperature of the fluid supplied by the utility unit can be adjusted.

After preheating, in embodiments utilizing microwave energy, product can proceed to the waveguide, for example, a waveguide located beneath the applicator. After heating in the microwave applicator unit, product can pass an infrared temperature sensor prior to entering the coolant unit. Alternatively, product can pass an RTD prior to entering the coolant unit. An RTD can be used in conjunction with an IR sensor or alone. Rates of heating within the waveguide can vary depending upon the length of the flow path within the waveguide, the power supplied the flow rate within the waveguide and the internal diameter of the tubing within the waveguide. The rate of heating within the waveguide can be empirically determined by the difference between the temperature of the material as it enters the waveguide and the temperature of the material as it exits the waveguide. In an example with a 42″ flow path of tubing within the waveguide, the following rates of heating, for a given power supply were estimated based on the heating characteristics of water (not empirically determined): 5 kW/63 degrees C. per second; 10 kW/126 degrees C. per second; 15 kW/189 degrees C. per second; 20 kW/251 degrees C. per second; 25 kW/314 degrees C. per second; 30 kW/377 degrees C. per second.

After product leaves the waveguide/applicator it is cooled. In an embodiment, the coolant unit includes a flat-plate heat exchanger. In an example the plate heat exchanger is designed for ease of sanitizing and prevention of residual contamination, for example by including removable plate connecting gaskets. In another example, the plates are provided as a disposable assembly to be discarded after processing is complete. In another embodiment, the coolant unit includes one or more shell and tube (also referred to as tube and shell) heat exchangers or other heat exchangers known in the art. In either embodiment, coolant fluid can be provided from a coolant loop of a utility unit.

In ultra short time heating and cooling, cooling can be most critical. The residence time in the microwave is relatively brief, for example, less than one-half second. The amount of time a product stays above the temperature at which it is stable often determines total product destruction. Thus, after an optimum time temperature profile is determined for product and, for example virus reduction, it is critical to control the cooling of the product so that the optimum is reached. Plate heat exchangers are one method of rapid cooling. Shell and tube heat exchange cooling is another method of rapid cooling. Another possible method of rapid cooling utilizes vacuum cooling. Still another method of cooling utilizes plate heat exchangers with a low viscosity fluid. Such a low viscosity fluid can provide an increased heat transfer coefficient and, therefore, more rapid cooling. Such cooling methods and systems can be used separately or in combination to provide maximum potential cooling strength and efficiency while limiting system pressure drops. Thus, optimum time/temperature profiles, that otherwise might be outside the parameters of the system, may be achievable.

It is optimum to begin cooling immediately after fluid exits the applicator/waveguide. In an embodiment in which plate heat exchangers are located some distance from the outlet from the waveguide/applicator, an additional intercooler can be provided as a pre-coolant unit. Such an intercooler can be designed to provide heat exchange cooling of fluid immediately after exit from the microwave applicator unit and prior to entering the coolant unit. The intercooler can be one or multiple parallel shell and tube heat exchangers. In an example, the unit is made of 316 stainless steel and is designed in a cylindrical shape. The flow path can run inside the cylinder, for example an approximately ⅛ inch ID flow path. Surrounding the flow path can be the cooling chamber. To maximize heat transfer, the cooling chamber can contain flow paths with sufficient pressure so that the cooling fluid has turbulent flow through the chamber. To further maximize cooling, the shell and tube can be jacketed. By jacketed we mean that a primary cooling chamber, through which coolant flows around the flow path, is surrounded by a secondary cooling chamber. The secondary cooling chamber can be supplied with coolant independently of the primary cooling chamber or, alternatively, utilize partially spent coolant after the coolant exits the primary cooling chamber. This may also be described as a double jacket. The secondary cooling chamber may provide additional cooling potential or may simply provide an outlet for spent fluid.

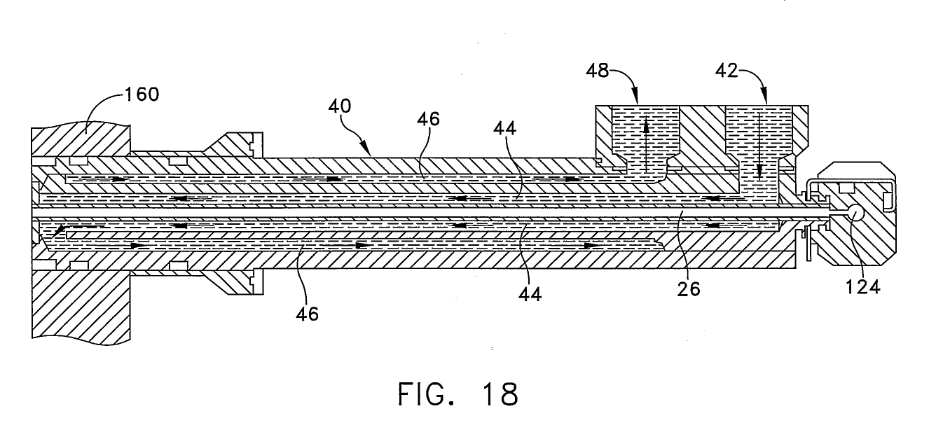

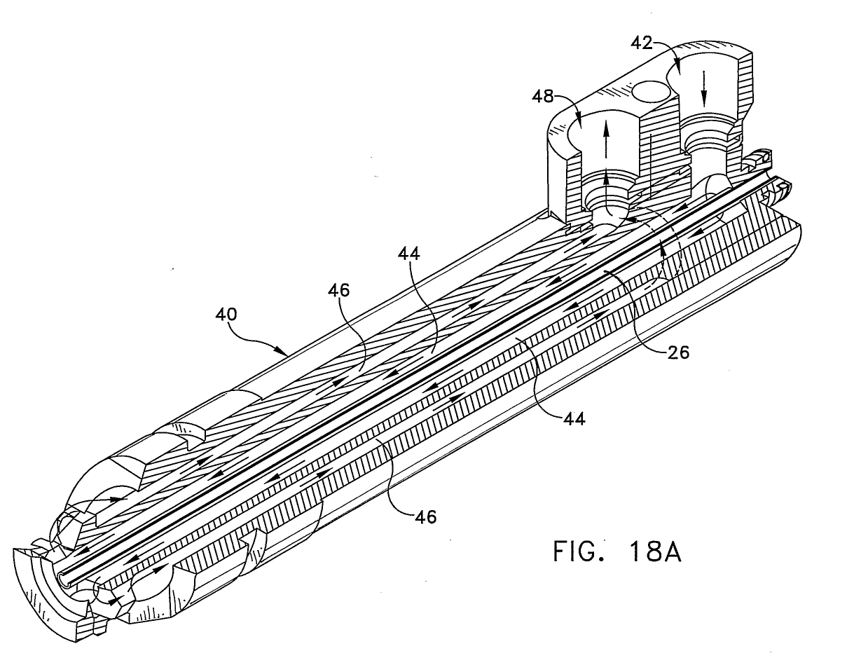

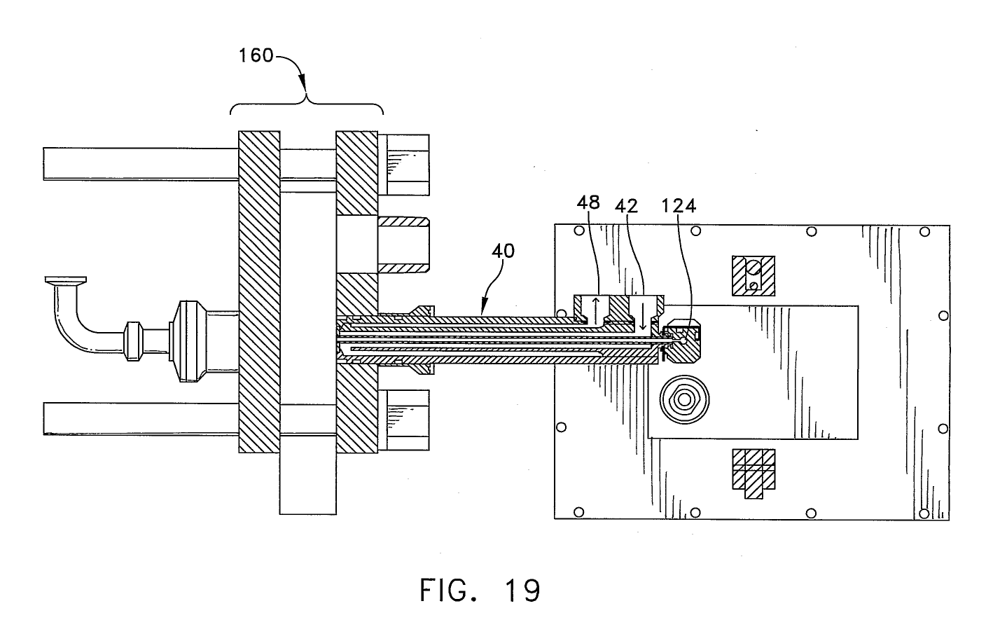

The following tables 1, 2 and 3, compare cooling rates and flow rates at particular peak temperatures. The cooling rates demonstrate the efficiency of a jacketed intercooler described above and shown in FIGS. 18, 18A and 19. For example, at flow rates of 425 liters per hour and peak temperature of 85 degrees C., the cooling rate within the intercooler was 429 degrees C. per second. Results at 425 L/hr were achieved with phosphate buffer. Results at 80 L/hr and 300 L/hr were calculated based on the 425 L/hr experimental results.

TABLE 1
Flow rate-80 liters per hour

| Peak Temp (degrees C.) | Cooling Rate (degrees C. per second) |
|---|---|
| 70 | 249 |
| 75 | 278 |
| 80 | 313 |
| 85 | 335 |
| 90 | 363 |
| 95 | 393 |
| 100 | 423 |

TABLE 2
Flow rate-300 liters per hour

| Peak Temp (degrees C.) | Cooling Rate (degrees C. per second) |
|---|---|
| 70 | 312 |
| 75 | 347 |
| 80 | 382 |
| 85 | 414 |
| 90 | 449 |
| 95 | 483 |
| 100 | 518 |

TABLE 3
Flow rate-425 liters per hour

| Peak Temp (degrees C.) | Cooling Rate (degrees C. per second) |
|---|---|
| 70 | 323 |
| 75 | 359 |
| 80 | 394 |
| 85 | 429 |
| 90 | 464 |
| 95 | 499 |
| 100 | 534 |

Specific targets and flow rates can be chosen depending on the product and contaminant properties.

In other embodiments, the intercooler can include, for example, cryogenic oil for rapid cooling. The intercooler can also be supplied with coolant by a separate chiller to provide fluid at lower temperature than is provided other components of the system.

In addition to use with particular products that may require rapid temperature decrease from peak temperature to survive, the intercooler can provide additional flexibility to the system. For example, if increased contaminant destruction or increased product survival is desired, the intercooler can be adjusted. Such adjustments can include changing the temperature of the coolant flowing within the intercooler or even stopping flow of coolant into the intercooler. Depending on the characteristics of the contaminant of concern, as compared to the product, this additional flexibility within the system and method may be required. Similarly, if a particular specification has been set for a time/temperature profile, the intercooler can add flexibility in achieving the goal. For example, if processing parameters have been set using a different or smaller system, the intercooler can help provide the system flexibility to match those parameters. Process parameters may also be set using a formula for calculating reaction rates, based on reaction rate constants, known as the Arrhenius constants for rates of reaction, for the product or contaminant. In any case, using two or more cooling apparatuses may subject the product to multiple cooling rates and times. For example, the cooling rate through an intercooler can be faster for a short period of time to bring the product below what might be a critical temperature. For example, product can flow through the intercooler in less than about 0.2 seconds. After product is below a particular temperature a slower cooling rate might be sufficient for example cooling in a plate heat exchanger for less than about 10 seconds.

In addition to use as an intercooler between the heater and another cooler, the intercooler design can be used as the sole or primary cooling mechanism. For example, by using one or more elongated jacketed (double jacket) or non-jacketed tube and shell heat exchangers to cool or as the primary cooling mechanism, for example multiple tube and shells set up in a parallel cooling configuration or one elongated tube and shell.

After product exits the cooling section or unit, an RTD can be used to provide an additional fluid temperature record. Fluid material can then either be sent to waste or product depending on the setting on shut-off valves leading to product and waste.

### Fluid Path

In certain embodiments, product flows into the fluid processing unit from either a separate remotely located inlet source module or from inlet containers adjacent or attached to the fluid processing unit. Product can flow in from the inlet through tubing of, for example, 1 inch ID or ½ inch ID. In an embodiment, product flows within the fluid processing unit through a 316 stainless steel flow path. In an example, the stainless steel flow path is about ⅛ to about ½ inch ID, for example ⅜ inch ID. Product flows in the ⅜ inch ID stainless steel flow path through a pump, such as an autoclavable, motorized pump, and then through a parameter check section that might include a bubble sensor employed external to the flow path. A portion, or all, of the stainless steel flow path can be replaced with a plastic or rubber flow path, for example of TEFLON, polypropylene, polyethylene or silicon. A bubble sensor can be employed, for example, around a silicon portion of the flow path. In this embodiment, product flows by a flow meter and pressure transducer and RTD before entering a pre-heat unit. In an embodiment using pre-heat plate heat exchangers, product flows from a ⅜ inch ID stainless steel flow path into a funneling cone shaped ⅜ inch ID sanitary adapter (e.g. a funnel plug) leading to the inlet of a pre-heat plate heat exchanger. The adapter is employed to allow leak-proof transition to the relatively wide inlet of the preheat heat exchanger while avoiding excessive system pressure fluctuation.

After flowing through the plate heat exchangers, product can exit the outlet from the plate heat exchanger, for example through a funnel plug or other adapter with a 60 degree taper to ⅛ inch ID. Alternatively, the taper can be to a wider diameter if the tubing leading to the microwave is widened. Adapters such as funnel plugs can be adapted to minimize pressure changes within the system as product flows from one diameter flow path to another to maintain flow velocity. In an example in which the exit of a funnel plug is ⅛ inch ID, product flows from the funnel plug into a ⅛ inch ID length of tubing leading to the applicator/waveguide. This ⅛ inch ID section may be optional depending on the sensitivity of the fluid at the temperature leaving the pre-heat. If the product is stable at the temperature, fluid may flow instead directly into a tubing of ID matching the ID of the tubing extending from the MW cover into the waveguide.

In an embodiment, fluid flows from the optional ⅛ inch ID tubing into the tubing of, for example, ¼ inch ID tubing within the waveguide. Fluid leaving the waveguide can pass into a T-fitting with, for example, a ⅛ inch ID flow path and an RTD inlet. An RTD at the T-fitting RTD inlet can be redundant to the IR sensor and can be used for calibrating the IR temperature sensor or, in an embodiment, to replace the IR temperature sensor. The flow path can include an intercooler (for example using a shell and tube heat exchanger or a jacketed shell and tube heat exchanger) to immediately begin cooling of fluid prior to entering, for example, cooling plate heat exchangers. The intercooler can be cylindrical in shape and have, for example, ⅛ inch ID tubing within. Connecting the intercooler to the T-fitting can be a ½ inch sanitary connection secured with a tri-clamp. Fluid can exit the intercooler through a funnel plug in reverse position from the funnel plug utilized at the exit from the preheat heat exchanger. Fluid enters the funnel plug through, for example, a ⅛ inch ID opening and exits the funnel plug through the tapered end into the inlet of the cooling plate heat exchanger. Alternatively, no funnel plug is used and a close connection is made between intercooler and plate heat exchanger. Upon leaving the plate heat exchangers, for example through a ½ inch sanitary connection secured by tri-clamp into ⅜ inch ID stainless steel tubing, fluid can pass an RTD to measure the temperature of the cooled fluid.

Subsequent to microwave heating, in certain embodiments, product cooling can be optimized by minimizing the length of flow path between the exit from the microwave applicator unit and the intercooler. Similarly, the length of flow path between the exit from the intercooler and the cooling plate heat exchanger can be minimized to optimize cooling efficiency. In addition, when more efficient cooling is occurring in the intercooler as compared to the subsequent coolant unit, for example a plate heat exchanger, increased length in the intercooler may be desirable. Alternatively, by elongating the intercooler design, the tube and shell can be used as a sole source of cooling.

It will be appreciated that the flow rate in the range of about 300 to about 450 liters per hour through tubing ranging in size from 1 inch ID or ½ inch ID to ⅛ inch ID or less requires adaptation to avoid excessive pressure changes within the system and maintain flow velocity. The series of sanitary fittings and adapters with tri clamps can be used to move within some range of ID. To minimize pressure variations, however, the funnel plugs and various other adapters can be used to optimize system performance.

In general, it is optimal to minimize system pressure drops. Funnel plugs can provide a mechanism to reduce system pressure drops, while also reducing resonance time through a section of the flow path. Funnel plugs can also serve as an adapter for use when moving fluid from a larger ID tubing section to a lower ID tubing section. Maximum pressure within the system is limited by the specifications of various components of the flow path. If pressure drops must occur, it is generally beneficial to focus such pressure drops at portions of the system in which maximum fluid velocity (resonance time) is most critical. For example, protein denature may be limited or non-existent at system pre-heat temperatures and times. In such a case, it may be optimum to minimize system pressure changes in the pre-heat section and allow longer residence times in the pre-heat section. To the extent protein denature occurs it will generally occur at a higher rate around peak temperature. To minimize denature, and maximize control over the time temperature profile, cooling efficiency should be optimized, for example by maximizing fluid velocity through the cooling sections. One method for maximizing fluid velocity in the cooling section is to minimize the ID of the tubing in that section. Although a system pressure drop may occur, system pressure is put to maximum advantage.

### Applicator/Waveguide

In an embodiment, the magnetron is a 2450 MHz magnetron supplied with 30 kW of power. A WR-430 waveguide can be used to direct microwaves to the product. The magnetron tube/waveguide assembly can be designed to operate remote from the power supply unit. The assembly can also include: a circulator to direct reflected power to a dummy load; a microwave tuner, such as an auto-tuner and a pressure window.

The inlet to the applicator/waveguide within the fluid processing unit can be through a T fitting on top of which can be an RTD port. The T fitting can be secured to the applicator cover (MW cover) that can be secured to the top of the applicator and can both cover the waveguide and secure the tubing within the waveguide.

The MW cover can be designed to either or both prevent microwave leakage from the applicator/waveguide and secure the tubing for product flow within the applicator/waveguide. The MW cover can be, for example, a ½ inch thick aluminum plate. The MW cover can be secured to the applicator via a series of clamps. The clamps can be either single clamps or connected in series. Connecting the clamps together allows efficient removal of the MW cover, provides a lever for undoing the clamps and provides and efficient mechanism to assure consistent, tight clamping. In an embodiment, the clamps, for example a series of two, three or more clamps, are connected by a solid material, for example metal or hard plastic bar through which a screw is placed to fasten each individual clamp (multi-clamps). In another embodiment a second bar is secured to another location on the clamps. One bar is used to release the clamps and another bar is used to lift the clamps off the MW cover.

The MW cover can be secured on top of the applicator by multi-clamps or a variety of other methods including other clamping methods. On the bottom side of the MW cover can be the tubing that is exposed to microwave energy. In an embodiment, the tubing is about 25 inches to about 100 inches in length, for example 50 inches. The internal diameter (ID) can be, for example about ⅛ inch ID to about ½ inch ID, for example ¼ inch ID. The diameter and length can be selected, for example, to accommodate desired increases or decreases in exposure times within the waveguide. In an embodiment, the tubing can be made of TEFLON. In another embodiment the tubing can be made with polyether ether ketone (PEEK).

Multiple cartridges may be required by a user and, therefore, it is beneficial to decrease the cost of each cartridge, whether or not the cartridges are disposable. In an embodiment, the cartridge includes the MW cover and tubing only, with preheat and cooling apparatuses separate. Reducing the components of the cartridge reduces the cost and, therefore, increases flexibility in disposal and replacements. The cartridge tubing can be wound in a variety of configurations depending on the desired microwave exposure, including vertically as shown in U.S. Pat. No. 5,389,335, or generally horizontally.

A mechanism that can be used to prevent leakage from the applicator/waveguide is an enlarged o-ring gasket installed into the top face of the microwave applicator. The o-ring gasket can be a metal filled silicon o-ring, for example, with a shielding effectiveness rating of 90 decibels at 10 gigahertz. The o-ring gasket can provide both a pneumatic seal and static shield for choking microwave energy within the waveguide. Conductive material within the o-ring can provide static grounding for the microwave frequency, for example 2450 MHz frequency. In addition, the o-ring can provide a pneumatic shield to prevent air leakage out of the waveguide.

A pressure window can be used to allow pressurization within a portion of the waveguide. The pressure window can be, for example, a quartz pressure window. The pressure window can also be made from TEFLON. The pressure window can be secured within the waveguide within a flange connection. To detect the potential for microwave leakage, the portion of the waveguide above a pressure window can be pressurized, at low pressure, for example, with air, or an inert gas. In an example, pressurization can be about 8 inches water. Air or inert gas can be directed into the waveguide above the pressure window. Pressurization within the waveguide above the pressure window allows for highly sensitive monitoring of proper system connections through detection of loss of pressure. Loss in pressure within the waveguide, for example from a loose connection or insecure fitting above the pressure window, can automatically shut the system down thereby preventing microwave leakage. To create the low pressure environment, required for sensitive leak detection, high and low pressure regulators can be used with pressure switches.

A pressure window can also prevent leakage material from falling into and contaminating the microwave field. Such contaminants have the potential to cause energy arcs which could damage the magnetron.

Redundancy for microwave leak detection can be built into some embodiments. For example, in addition to pressurization monitoring within the waveguide, microwave leak detectors can be used. Further redundancy can be provided using proximity sensors to determine that system components are in place and fastened correctly. For example, a proximity sensor detector may be positioned within the applicator and below the faceplate.

### Supervisory Control Unit

The supervisory control unit can be physically attached to the fluid processing unit so that the operator stands adjacent to the unit during operation. The supervisory control unit can also be a separate module operated remotely from the fluid processing unit.

The supervisory control unit user interface can be programmed using supervisory control and data acquisition software (SCADA software). The SCADA software communicates with the programmable logic controller (PLC) through a serial bus. SCADA software provides instructions to the PLC, for example, a Siemens 505 Series. Multiple input/output racks are controlled by the PLC. In a particular example of three input/output racks, one controls the fluid processing unit, one controls the utility unit and one controls the power supply unit. All communications to the PLC can be through a network, such as a Profibus network over a network cable, or wireless network, thus allowing multiple modules, in remote locations, to be operated by the supervisory control unit (which may be remote or physically attached to the fluid processing unit).

It will be appreciated to those skilled in the art that although a microwave system may provide the most efficient rapid heating, many aspects are applicable to other heating systems. Such an alternative heating system may utilize plate exchangers and/or steam heating for all heating requirements. Many aspects will be applicable to such a heater including the modularity of the system, features relative to reducing pressure changes within the system and methods and devices used to cool.

### DETAILED DESCRIPTION OF THE DRAWINGS

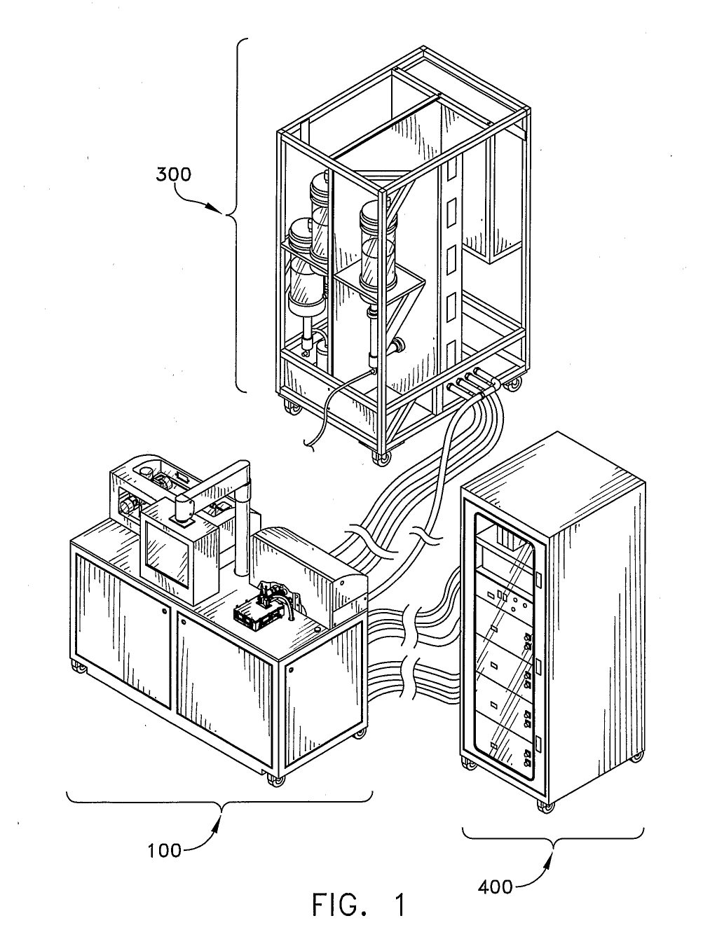

FIG. 1 is an overview of an embodiment including a fluid processing unit 100, utility unit 300 and power supply unit 400. Each unit is connected by a series of plumbing and electrical connections. Each unit can be on wheels or can be otherwise designed for easy movement from one location to another.

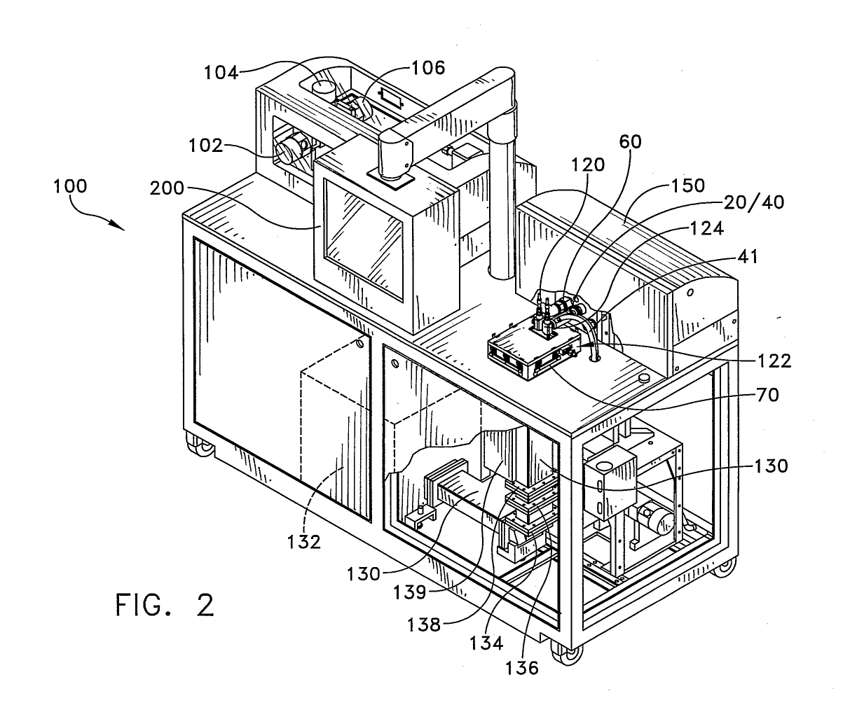

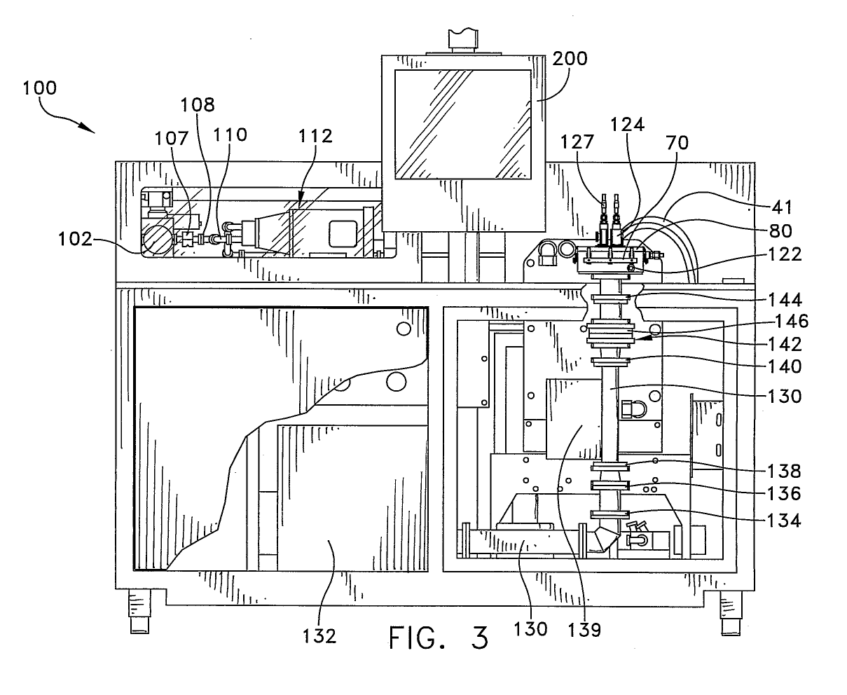

FIG. 2 is a perspective view of a fluid processing unit 100. Product passes through one of inlet valves 102, 104, 106 and into motorized pump via 316 stainless steel flow path. Plate heat exchanger is within cover 150. Product exits the pre-heat plate heat exchanger (within cover 150) and can, for example, pass through either a sanitary elbow with a thermowell, or as shown in FIG. 2, a T-fitting 127 and into the applicator 122/waveguide 130. Product exits the applicator 122/waveguide 130 through a T-fitting 124 through an intercooler 20 and into the cooling plate heat exchanger (under cover 150). The waveguide 130 begins at the magnetron box 132 and makes a 90 degree connection to the vertical portion. Flanges 138 and 140 (FIG. 3) provide inlets for coolant. An auto-tuner is attached to the waveguide (in box 140) with components extending into the waveguide. The waveguide ends at the top of the applicator 122 upon which is the MW cover 80 with multi-clamps 70. Also shown is screen 200 for supervisory control unit, in this embodiment physically attached to the fluid processing unit 100.

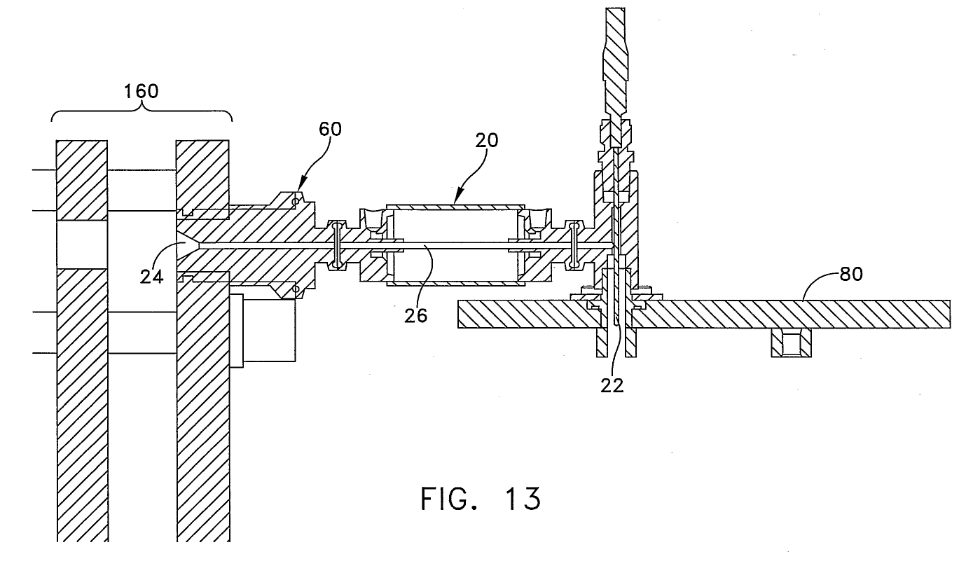

FIG. 3 is a front view of a fluid processing unit 100. Product passes through inlet valve 102 and into motor/pump 112 via 316 stainless steel flow path 110 with sanitary fittings 108 and bubble sensor 107. Product exits a motorized pump 112 and passes through the 316 stainless steel flow path between the pump and the pre-heat plate heat exchanger. Product can exit the pre-heat plate heat exchanger through, for example, a sanitary elbow with a thermowell or, as shown in FIG. 3, through a T-fitting 127 entry into the applicator 122/waveguide 130. Product exits the applicator/waveguide through a T-fitting 124 through either a jacketed intercooler 40 or non-jacketed intercooler 20 (see for example FIG. 13 using non-jacketed intercooler and funnel plug connection) and into the cooling plate heat exchanger 160 (see for example FIG. 13). Intercooler is fed, for example cold water or glycol, and relieved of spent coolant through lines 41. The waveguide 130 begins at the magnetron box 132 and has 90 degree elbow leading to the vertical portion. Also shown are flanges 134, 136, 138, 140, 142 and 144. Flanges 138 and 140 provide inlets for coolant such as water or a water glycol mixture or other cooling fluids. An auto-tuner is attached to the waveguide (in box 139) with components extending into the waveguide. The waveguide ends at the top of the applicator 122 upon which is the MW cover 80 with multi-clamps 70 and downwardly extending tubing above the flange 144. The pressure window flange 142 holds the pressure window in place. Above the pressure window flange 142 is an air inlet for pressurizing above the pressure window. Also shown is screen 200 for supervisory control unit, in this embodiment physically attached to the fluid processing unit.

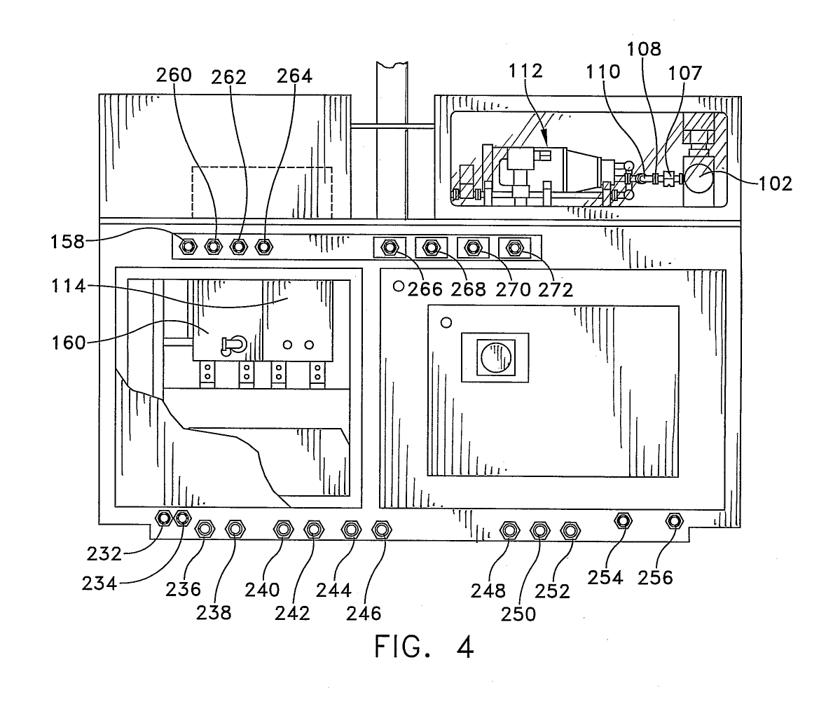

FIG. 4 is a partial rear view of the back of a fluid processing unit. Product passes into the system through one of the inlet valves 102 and into motor/pump 112 via the stainless steel portion of the flow path 110 with sanitary fittings 108 and bubble sensor 107 employed at a TEFLON portion of the flow path. Product exits a pump 112 and passes through the stainless steel flow path between the pump and the pre-heat plate heat exchanger 114. Product exits the pre-heat plate heat exchanger 114 and enters the applicator 122/waveguide 130. Product exits the applicator 122/waveguide 130 and enters the cooling plate heat exchanger 160. Product exits the cooling plate exchanger 160 and either passes to waste, through waste line outlet 232 or is kept as product through product line outlet 234. Adjacent to waste line outlet 232 and product line outlet 234 are utility supply and return lines connected to the utility module, including coolant supply inlet 236, coolant return 238, hot water supply 240 for pre-heat heat exchanger 114, hot water return 242, cold water supply 244 and cold water return 246. Connectors for top mounted components (258, 260, 262, 264, 266, 268, 270 and 272) include RTD ports for the RTD that measures the temperature at the inlet to the preheat 258, exit from the preheat 260, exit from the microwave 262 and exit from cooling section 264. Also shown are signal connections for media pressure transmitter 266, media flow indicator 268, inlet valve position signal 270 and media pump power switch 272. Control panel 274 is the electronic control enclosure. Also shown are attachment points (248, 250, 252, 254 and 256) for power supply lines, including high voltage lines and signal line, air supply lines (for actuating inlet and outlet valves and pressurizing the waveguide) and network lines.

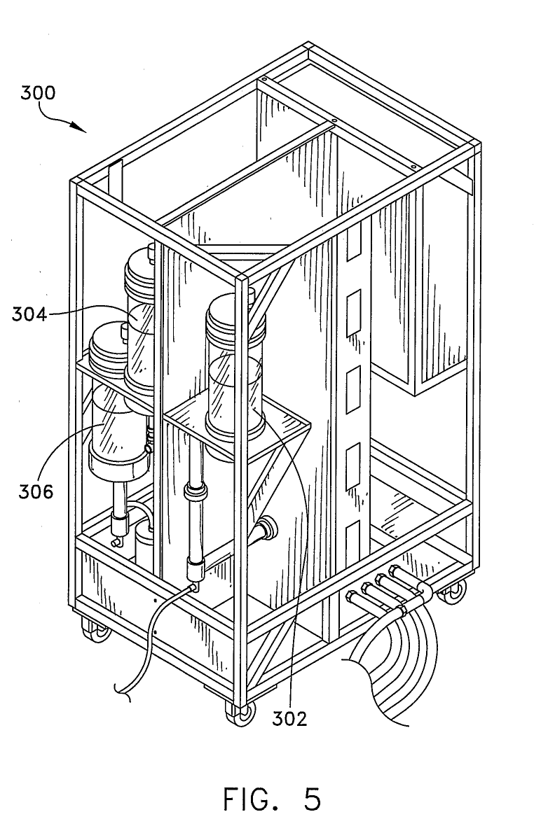

FIG. 5 is a perspective view of a utility skid that can supply utilities including hot water, coolant and cold water to the system. In an example, the utility skid includes a cold water reservoir 304 and coolant reservoir 306 on one side and a hot water reservoir 302 on the other side. Hot water, coolant and cold water flow to and from the fluid processing unit through pumps, tubing, flow meters and RTD's all of which can be monitored and controlled remotely.

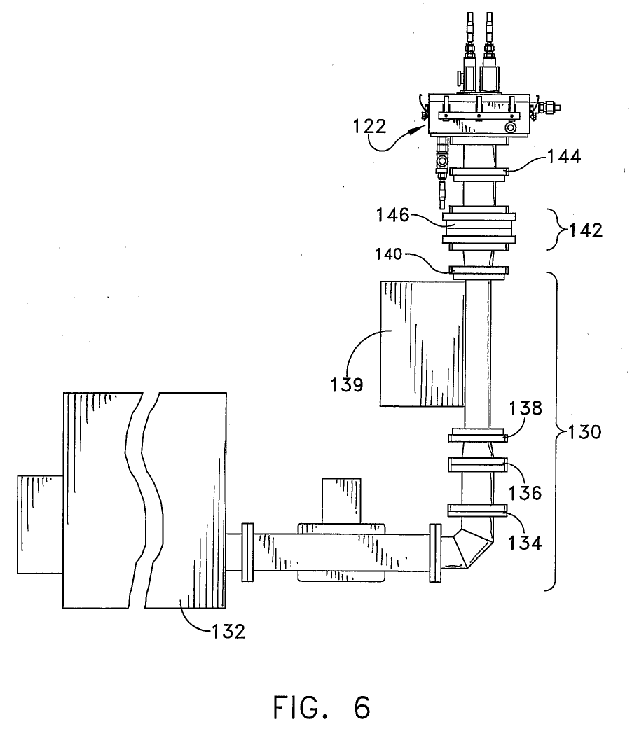

FIG. 6 is a schematic drawing of a waveguide 130 starting at a magnetron box 132. Microwaves are generated by a magnetron within the magnetron box 132 and are propagated to the ninety degree angle and up toward the applicator 122. The waveguide 130 includes several flange connections including 134, 136, 140, 142 and the top flange 144. Within flange 142 is the pressure window 146. Microwave auto-tuner box 139 is attached to the waveguide 130 and includes projections into the waveguide 130 that automatically adjusts the microwave field.

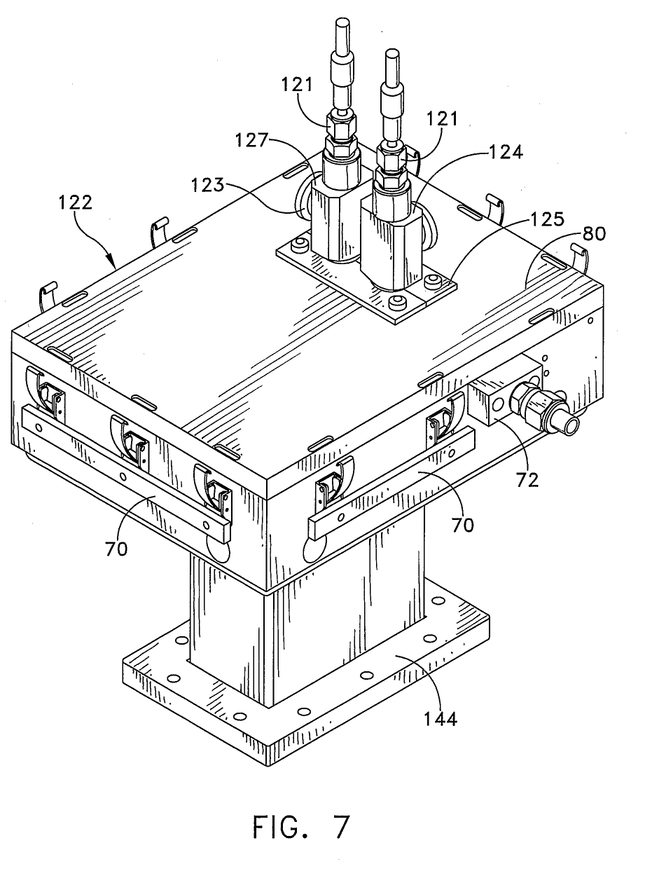

FIG. 7 is a perspective view of a microwave applicator 122 and top flange 144 with MW cover 80 in place. Multi clamps 70 on all four sides of the applicator are in the non-engaged position. Infrared sensor secured in block 72 provides temperature measurements for fluid after heating in the microwave. When secured to the waveguide, the top flange 144 can be within the cabinet. Two specialized T fittings 124/127, one inlet 127 and one outlet 124 include three separate sealing surfaces including a nut/ferrule connection 121 on top that can be used to secure an RTD. A sanitary fitting 123 on the side can provide either fluid inlet or outlet and a combination of sanitary and flange fittings 125 on the bottom can secure the T-fitting to the MW Cover 80. Product can enter the applicator 122/waveguide 130 from the pre-heat heat exchanger through the inlet T fitting 127 and can exit the applicator 122/waveguide 130 through the outlet T fitting 124 into a non-jacketed intercooler or jacketed intercooler.

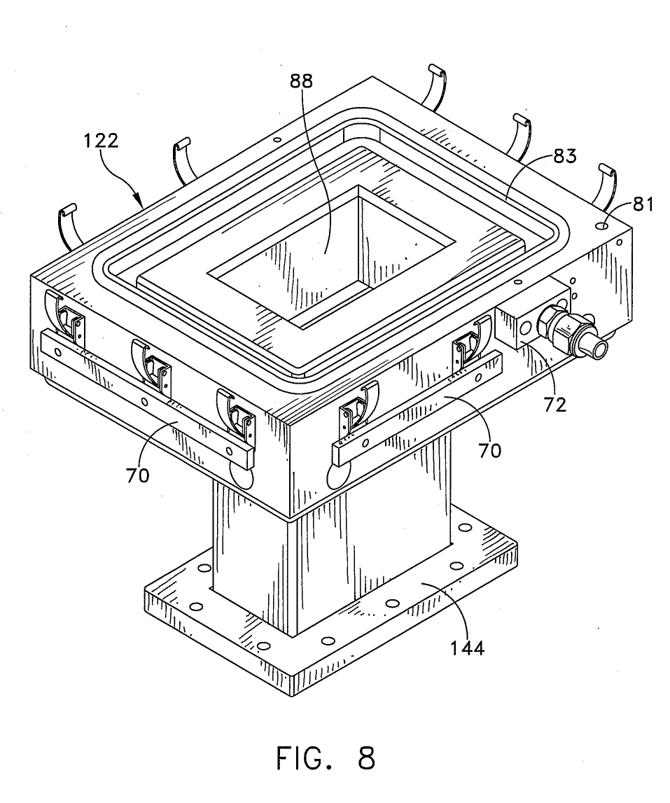

FIG. 8 is a perspective view of a microwave applicator 122 without MW cover 80 and with top flange 144 for connecting the applicator 122 to the waveguide 130. Multi clamps 70 on all four sides of the applicator 122 are in the non-engaged position. Infrared sensor inlet block 72 is also shown. Proximity sensor 81 provides redundant protection against an inadequately secured MW cover 80. O-ring gasket 83 holds air pressure in for presence sensing air pressure switch and provides a conductive electrical contact for grounding, electromagnetic inductive shielding and microwave choke. When secured to the waveguide 130, the top flange 144 sits within the cabinet and out of view. Opening 88 is at the top of the WR-430 waveguide 130 and is the space in which, for example, microwave transparent tubing is placed for exposure to microwave energy.

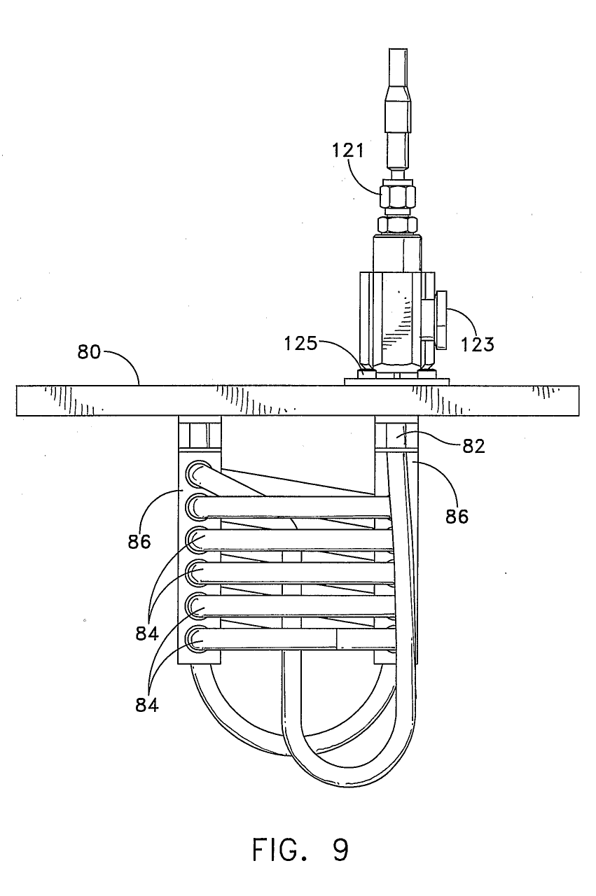

FIG. 9 is a schematic side view of an aluminum MW cover 80 with outlet 82 and tubing 84 extending downwardly from the MW cover 80, wound generally horizontally through preferably microwave transparent plastic holders 86 secured to the MW cover 80. Various tubing lengths are possible depending upon the time/temperature profile and, therefore, the time required within the waveguide. Also shown is specialized T-fitting 124 leading into the tubing 84 including three separate sealing surfaces including a nut/ferrule connection 121 on top used to secure the RTD. A sanitary fitting 123 on the side can provide either fluid inlet or outlet and a combination of sanitary and flange fittings on the bottom 125 can be used to secure the T-fitting to the MW cover 80.

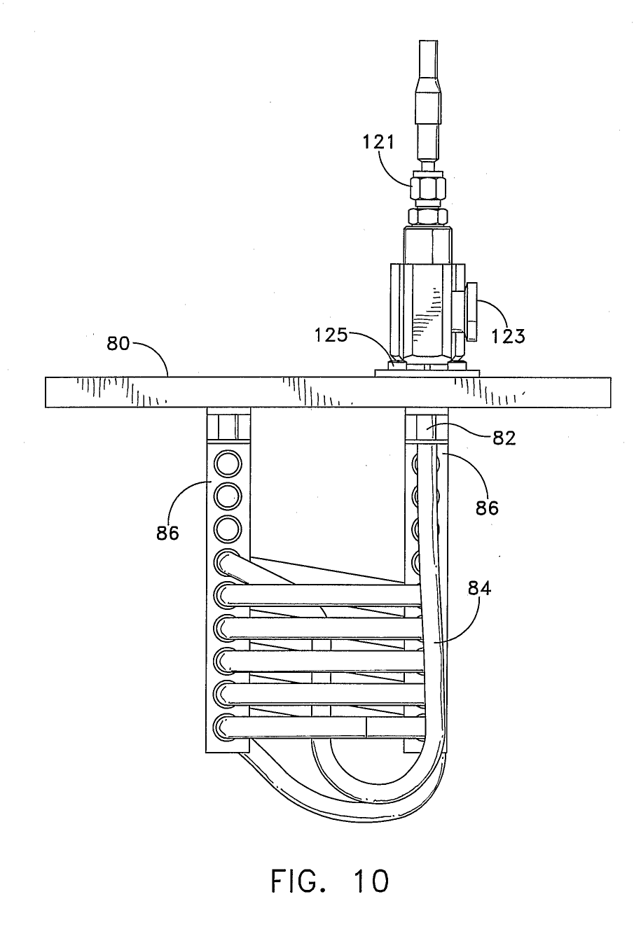

FIG. 10 is a schematic side view of an alternative embodiment in which the tubing 84 is wound lower on the microwave transparent plastic holders 86.

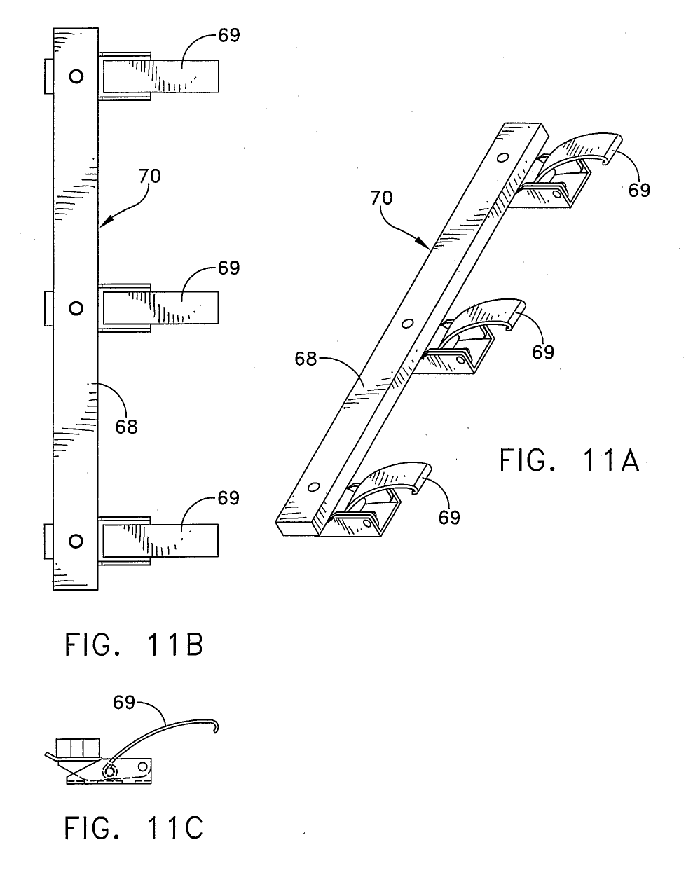

FIGS. 11A, 11B and 11C show a method and device for securing the microwave cover to the applicator using a multi-clamp 70 with a bar 68 secured to a clamp 69 for ease of use and tight connections.

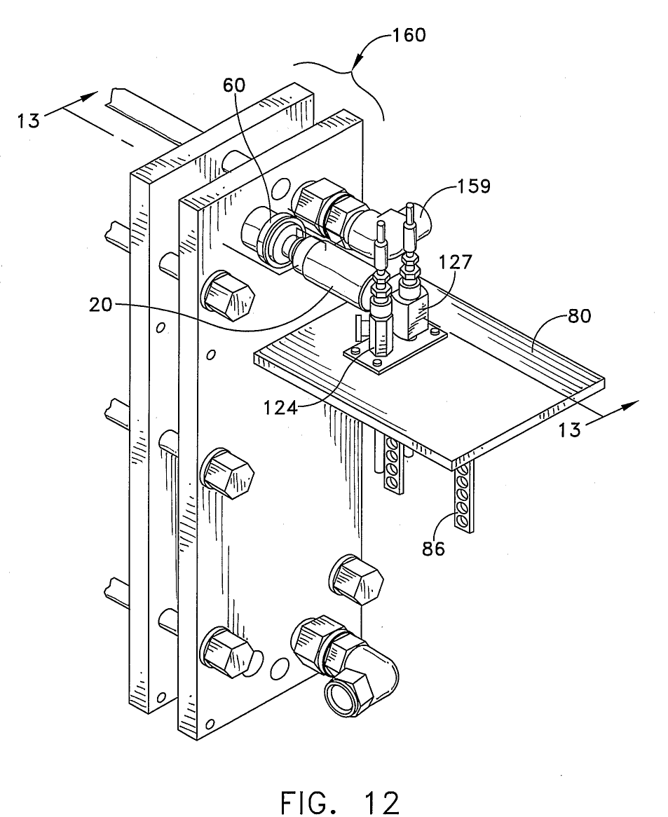

FIG. 12 is a perspective view of a MW cover 80 with microwave transparent plastic tubing holders 86 with MW cover connected to a cooling heat exchanger 160. An intercooler leads to the entry to the cooling plate heat exchanger 160 through a reverse position funnel plug 60. Special T fittings 127/124 provide connections from the applicator to the intercooler 20. Downwardly extending microwave transparent plastic holders 86 are shown without tubing. Cooled fluid, such as cooled product, exits the cooling plate heat exchanger 160 through an exit port.

FIG. 13 is a cross-sectional drawing of an example of a non-jacketed intercooler 20 positioned between the outlet 22 from the waveguide/applicator and the inlet 24 to the cooling plate heat exchanger 160. Product can be heated first by a pre-heat heat exchanger and then pass into the tubing extending into the waveguide from the MW cover 80. After entering the tubing through the MW cover 80 the product can be heated to peak temperature. It may be desirable to begin cooling immediately upon exiting the tubing. Product can exit the tubing through a T fitting 124 and then enter the fluid pathway 26 within the intercooler 20. In this example, product enters the funnel plug 60 prior to entering the cooling plate heat exchanger 160.

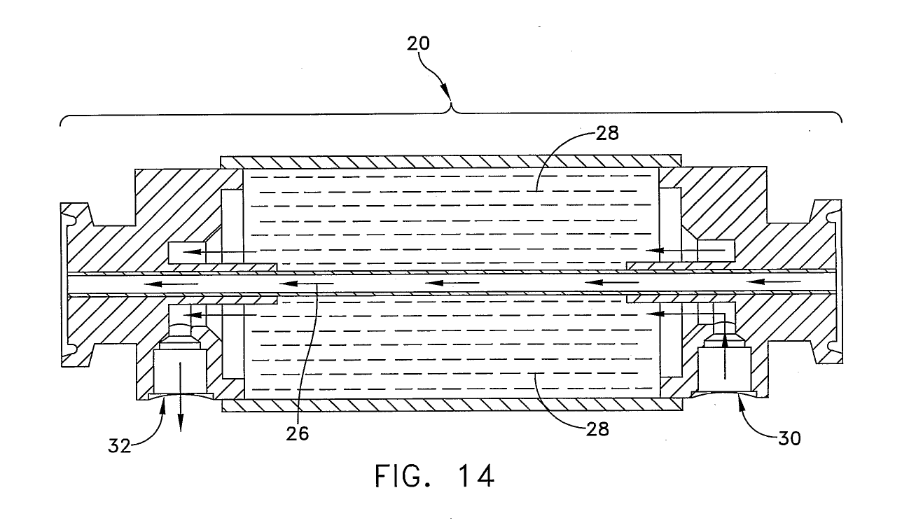

FIG. 14 is an enlarged cross-sectional drawing of a non-jacketed intercooler 20 including fluid pathway 26 surrounded by hollow space 28 through which coolant fluid can be supplied from a coolant loop. Coolant flows into the intercooler 20 through an inlet 30 and into the hollow space 28 surrounding the fluid pathway 26. Coolant flows through the space 28 and out through the outlet 32.

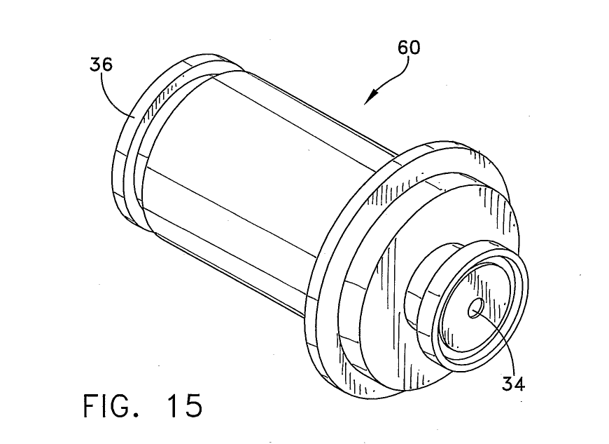

FIG. 15 is a front perspective view showing a funnel plug 60 with a one-eight ID enter/exit port 34. The funnel plug 60 can be constructed of 316 stainless steel. The flow path through the middle of the funnel plug 60 can become part of the flow path of the fluid processing unit 100. The ⅛ inch ID port 34 end of the funnel plug can be secured using sanitary fitting to connecting sections of the fluid path of the fluid processing unit 100. In an embodiment, funnel plugs can be used to adapt to the change in ID, such as: a) at the outlet from the pre-heat plate exchanger; and b) at the inlet to the cooling heat exchanger. Funnel plugs can also help reduce resonance time through portions of the flow path. An outlet from the pre-heat plate heat exchanger can be a larger ID space than the entrance to the flow section leading to the applicator/waveguide. The tapered end 36 of the funnel plug 60 can guide fluid flow from the outlet of the pre-heat to the ⅛ inch ID flow path leading to the applicator/waveguide. In another embodiment, the funnel plug 60 includes a ¼ inch ID flow path adapted to guide fluid into a one-quarter inch flow path leading to the applicator/waveguide.

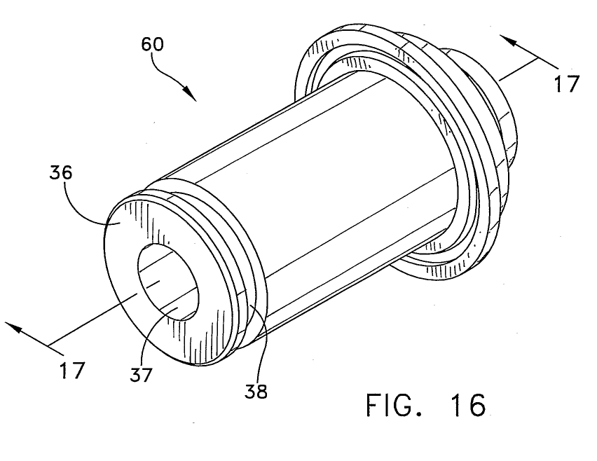

FIG. 16 is a rear perspective view showing the tapered end 36 of a funnel plug 60 including sixty degree taper 37 and gasket 38.

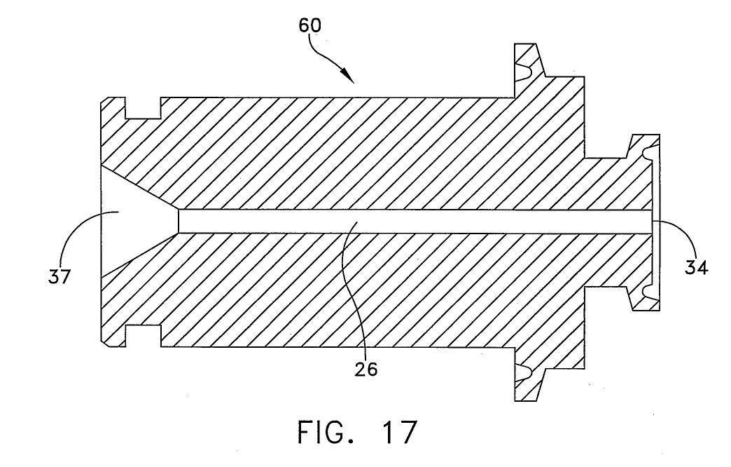

FIG. 17 is a cross-sectional drawing of a funnel plug 60 showing the sixty degree funnel section 37 opposite the port 34 and product pathway 26.

FIG. 18 is a cross-sectional drawing of a jacketed intercooler 40 showing product flow path through a 1.368 inch diameter, 316 stainless steel, jacketed intercooler 40, with an approximately one-eighth inch diameter length of flow path 26 tubing within. In this embodiment, the total flow path 26 length is 8.05 inches. The length of flow path actually within the shell 205 is 7.44 inches, thereby minimizing the length of flow path not subject to cooling. Coolant flows into the inlet 42 of the shell 44 and out of the shell 44, through openings, into the jacket 46 and exits from the jacket 46 through the outlet 48 to be cooled and re-circulated. The jacket 46 provides, in essence, a double shell in which the partially spent coolant can be reused and, thereby, may reduce heat gain on the outside wall. In shell 44 and jacket 46 the arrows indicate the direction of coolant, for example glycol, flow. In flow path 26, the arrows indicate the direction of product flow. Although exact dimensions of this embodiment are provided, this is not meant to be limiting as the possible dimensions are variable. For example, the flow path 26, shell 44 and jacket 46 can be dimensioned for turbulent flow thereby maximizing heat transfer. In this drawing, for example, the cooling shell 44 has an internal diameter of 0.468 inches and an outer diameter of 0.70 inches and the cooling jacket 46 has an internal diameter of 0.937 inches and an outer diameter of 1.368 inches.

FIG. 18A is a partial cross-sectional drawing of a jacketed intercooler 40 shown in FIG. 18. Arrows show the direction of coolant flow.

FIG. 19 is a plan view showing placement of the jacketed intercooler 40 shown in FIG. 18 with the addition of the illustration of a direct connection into the cooling plate heat exchanger 160. Product flows out of the microwave applicator T fitting outlet 124 and into the pre-coolant unit path 26. Using a non-jacketed intercooler, with direct, or close, connections into the cooling plate heat exchanger 160, similar rapid cooling may be achieved.

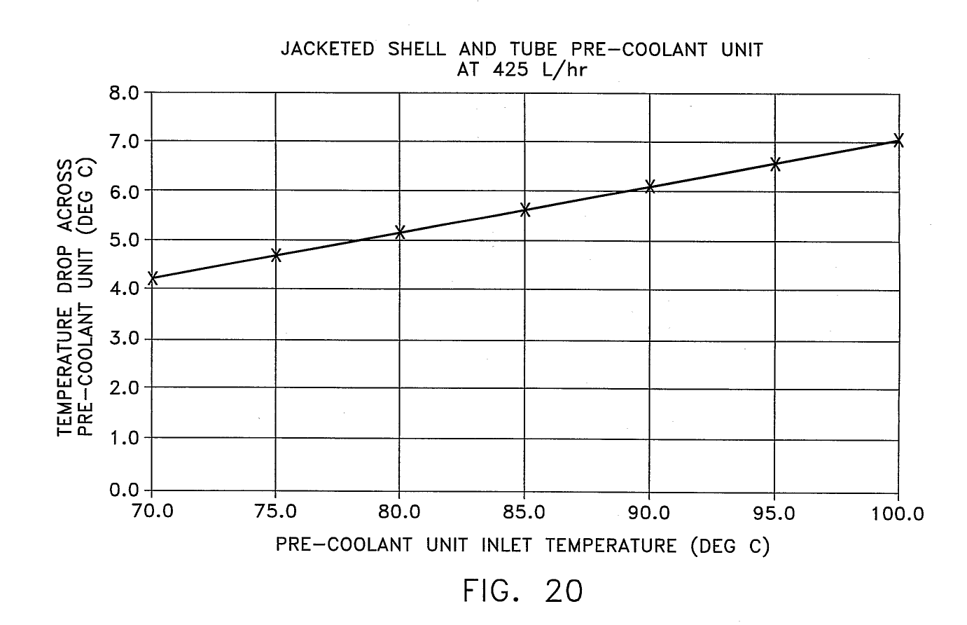

FIG. 20 is a graphical representation of cooling efficiency within the jacketed intercooler described in FIGS. 18, 18A and 19 flowing at a rate of 425 liters per hour. The x axis is the temperature of product entering the intercooler and the y axis is the amount of temperature reduction that occurs within the intercooler described in FIGS. 18, 18A and 19. For example, product entering at 100 degrees C. will exit at 93 degrees C. Product entering at 75 degrees C. will exit at just under 70 degrees C. It may also be possible to achieve similar results with a non-jacketed intercooler dimensioned and placed within the flow path as shown in FIGS. 18, 18A and 19.

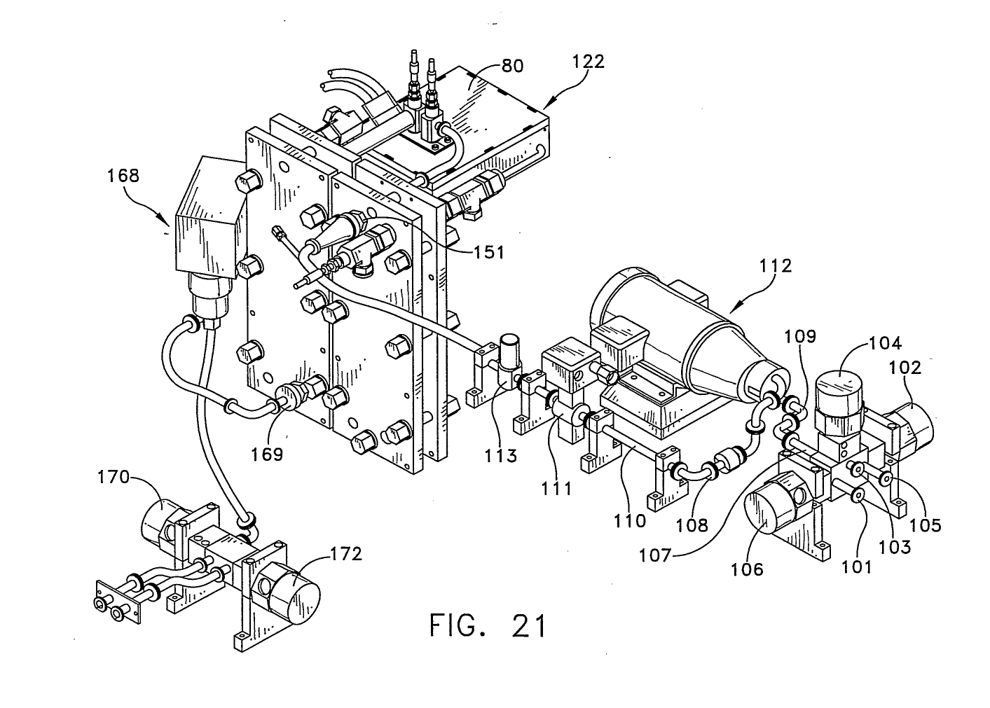

FIG. 21 is a perspective view of a flow path leading from inlet ports 101, 103, 105 and inlet valves 102, 104, 106 through 316 stainless steel flow path including bubble sensor 107 sanitary fittings and elbows to motorized pump 112 and again to 316 stainless steel flow path 110 with sanitary fittings 108 with elbows 109 to flow sensor 111 and pressure sensor 113 and to sanitary adapter 151 inlet of pre-heat heat exchanger. It is possible to replace stainless steel in areas of the flow path with other material such as TEFLON tubing. The flow path exits the applicator 122 and the cooling heat exchanger through outlet 169. Back pressure valve 168 prevents boiling in the flow path. The product exits through outlets controlled by outlet valves 170 and 172.

### EXAMPLES

### Example 1

In a particular example utilizing a one-quarter inch internal diameter (ID) 50 inch cartridge (the portion of the fluid processing unit in which microwave exposure occurs) and a flow rate of 425 liters per hour, and 28 kW of power, thermal destruction of simian virus 40 (SV40) was calculated, using MathCad, for various phases of heating and cooling.

Time in the preheat section of the fluid processing unit was 3.956 seconds. After exiting the preheat section, product flows through a preheat hold tube, of one-quarter inch ID and length of 10 inches, prior to entering the applicator/waveguide. Time in the preheat hold tube was 0.136 seconds. The total preheat time was, therefore, 4.092 seconds. Total time of microwave exposure was 0.341 seconds. From the applicator/waveguide fluid enters a 10 inch long intercooler with ID ⅛ of an inch. Product is within the intercooler for approximately 0.017 seconds. After leaving the intercooler fluid enters the cooling plate heat exchanger. Fluid exits the cooling plate heat exchanger after approximately 5.56 seconds.

Log base 10 reduction (log reduction) of SV 40 within the system was calculated from known Arrhenius reaction rate constants of SV 40. Log reduction in various portions was as follows: preheat section 1.445×10⁻³; applicator/waveguide 1.825; intercooler 0.659; cooling heat exchanger 6.74; total log reduction 9.225.

### Example 2

Comparison was made between inactivation of glucose oxidase and beta-galactosidase within a 5 kW, 2450 MHz microwave system and 30 kW, 2450 MHz microwave system. Results show similar destruction profiles.

### Beta-Galactosidase Assay

1.0 ml, 100 mM sodium acetate buffer, pH 5.0 and 0.5 ml, 20 mM o-nitrophenol-β-D-galactoside (ONPG) solution was pipetted into glass tubes and warmed to at 37 degrees C. One tube was prepared for each sample to be assayed. After warming the solution for 5-10 minutes the assay of the first sample was initiated by adding 100 ul of solution to be assayed, vortexing up and down 5 times, and immediately returning the tube to the 37 degrees C. incubator. Subsequent samples were initiated every 20 seconds. The reaction was stopped after 15 minutes by adding 2.0 ml, 0.2 M Na Carbonate solution and vortexing at room temperature. After all assays were stopped, absorbance (420 nm) was read at a rate of one assay every 20 seconds, in the same order in which the assays were initiated.

### Glucose Oxidase Assay

60 ml, pH 5.7 MES-sodium buffer, 12 ml, 15% Beta-D-glucose solution, 0.6 ml, 0.5% AA solution, 0.6 ml, 40 mM EHSPT solution and 0.6 ml, 1.8 mg/ml peroxidase solution were mixed together and 3 ml was transferred into glass tubes and warmed to 37 degrees C. One tube was prepared for each sample. After the solution warmed for 5-10 minutes the assay of the first sample was initiated by adding 50 ul of solution to be assayed to the tube with the assay solution, vortexing up and down 5 times and immediately returning the tube to the 37 degrees C. incubator. Subsequent samples were initiated every 20 seconds. Seven minutes after initiating each assay, the absorbance at 555 nm was determined in the spectrophotometer and the result recorded.

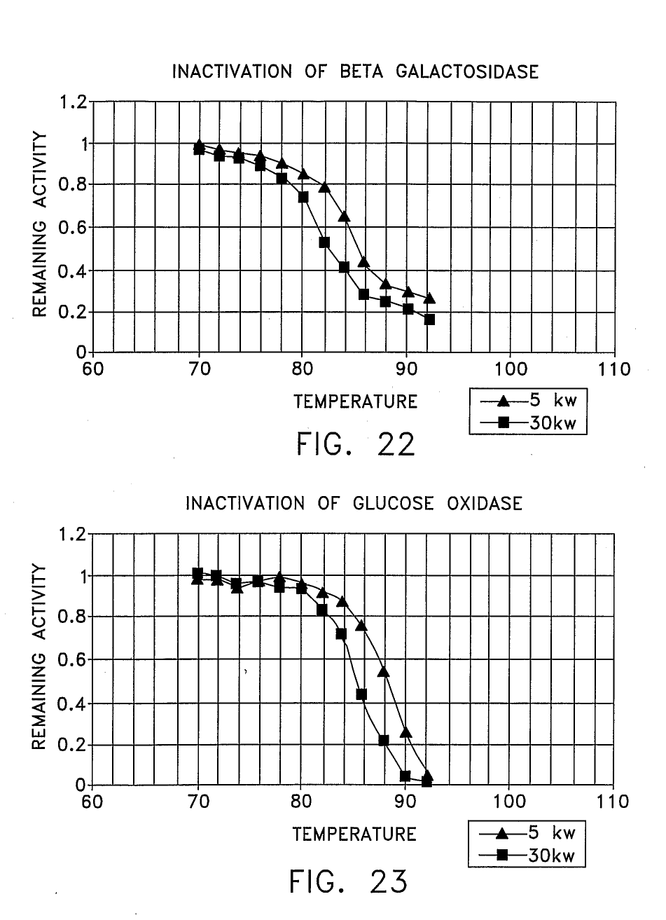

FIGS. 22 and 23 are graphical representations of experimental results. Results show that in the cases of both beta galactosidase and glucose oxidase the 5 kW system provides a more favorable destruction profile. These experiments were conducted using a non-jacketed intercooler, connected to a cooling plate heat exchanger through a funnel plug, as shown in FIGS. 12, 13 and 14.

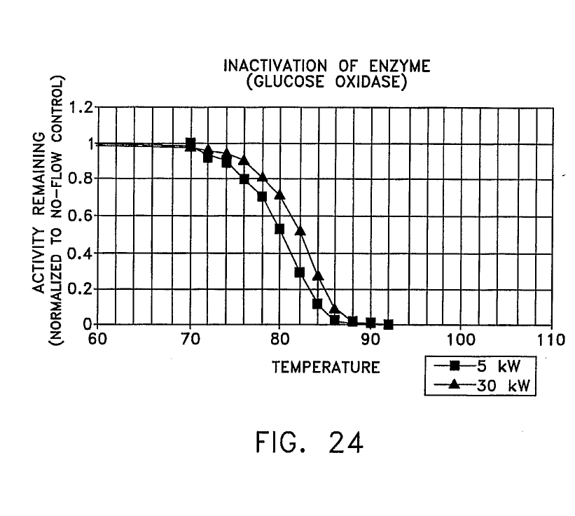

FIG. 24 shows results from experiments with glucose oxidase and a jacketed intercooler shown in FIGS. 18 and 19. As shown in those figures, the jacketed intercooler was extended to the cooling plate heat exchanger without the use of a funnel plug so that the portion of the flow path, between the exit from the microwave and before the cooling plate heat exchanger, that is not undergoing cooling heat exchange is minimized. Results show the improvement relative to the 5 kW, 2450 MHz system to the extent that the preservation of glucose oxidase was greater, at target relevant temperatures, using the 30 kW, 2450 MHz system with the jacketed intercooler compared to the 5 kW, 2450 MHz.

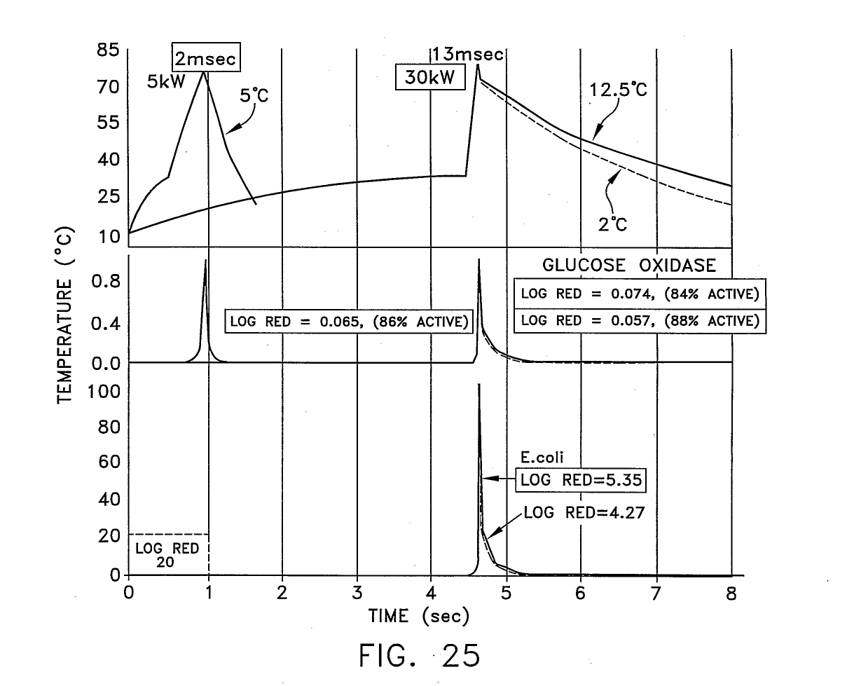

FIG. 25 graphically illustrates results using a jacketed intercooler jacketed shell and tube heat exchanger) to compare log reduction of E. coli with inactivation of glucose oxidase using coolant at 12.5 degrees C. as compared to coolant at 2 degrees C. Glucose oxidase activity was improved 4% at the expense of a 0.88 loss of log reduction of E. coli. FIG. 25 also illustrates the time/temperature profile achieving equivalent inactivation/product recovery using a smaller, in this example 5 kW, 2450 MHz, system. This figure further demonstrates the importance of rapid and immediate cooling; although heating and cooling profiles for the 5 kW, 2450 MHz and 30 kW, 2450 MHz systems appear dissimilar, the destruction and recovery rates are similar. This is largely due to the rapid cooling from peak temperature achieved using an intercooler between the exit from the waveguide and the entrance to the cooling plate heat exchanger.

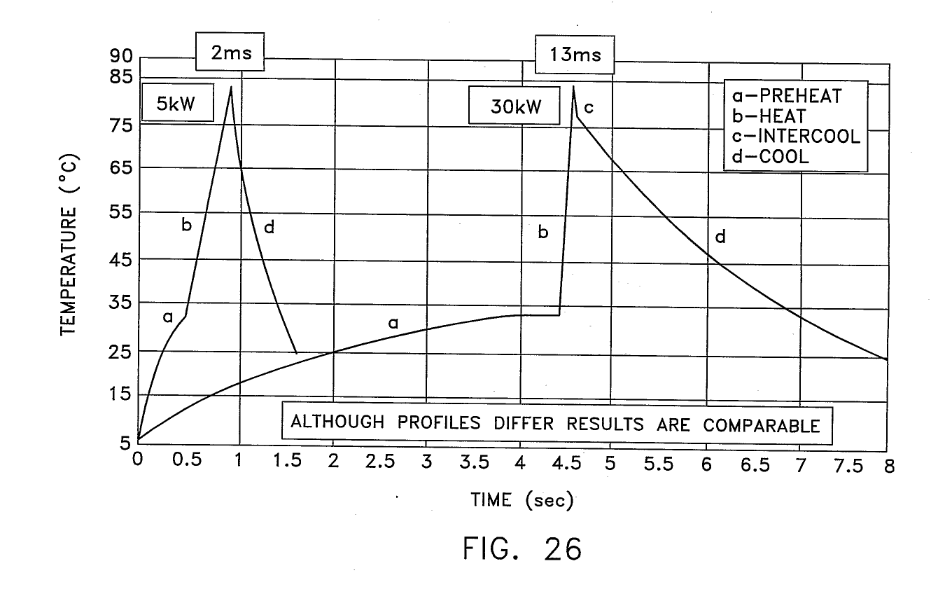

FIG. 26 graphically illustrates typical heating and cooling profiles achieved using a jacketed shell and tube heat exchanger illustrated in FIGS. 18 and 19. With a 30 kW power supply and a 2450 MHz generator, product is pre-heated for approximately 4.3 seconds to a pre-heat temperature of approximately 33 degrees C. (marked “a” on 30 kW graph). Product then enters the microwave applicator and is heated to peak temperature within milliseconds (marked “b” on 30 kW graph). Product then enters the intercooler where it is cooled for approximately 13 milliseconds (marked “c” on 30 kW graph) before entering the plate heat exchanger cooling unit (marked “d” on 30 kW graph). Also shown in FIG. 25 is the heating and cooling curve using a 5 kW system in which the equivalent reaction (inactivation of E. coli and maintenance of glucose oxidase activity) occurs. By providing a 30 kW system that can achieve a similar reaction as compared to a smaller, for example 5 kW, 2450 MHz system, it is possible to use the smaller, less expensive, system for experimentation and then use the 30 kW system for production scale-up using the time/temperature requirements determined from experimentation with the smaller system. Each product/contaminant combination will have specific conditions required for inactivation/activity maintenance. Predicting those conditions using the smaller system, for example by injecting a small volume of product into a flowing stream and obtaining contaminant destruction and product recovery information, is another aspect. It will be appreciated that times and temperatures can be varied and the exact temperatures and times described are for the purposes of this example only.

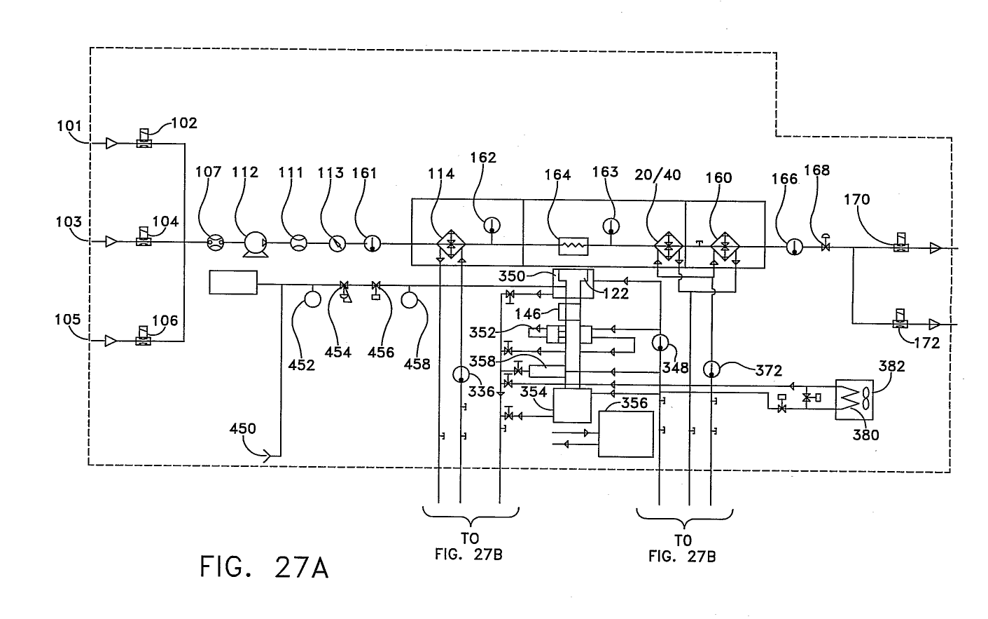

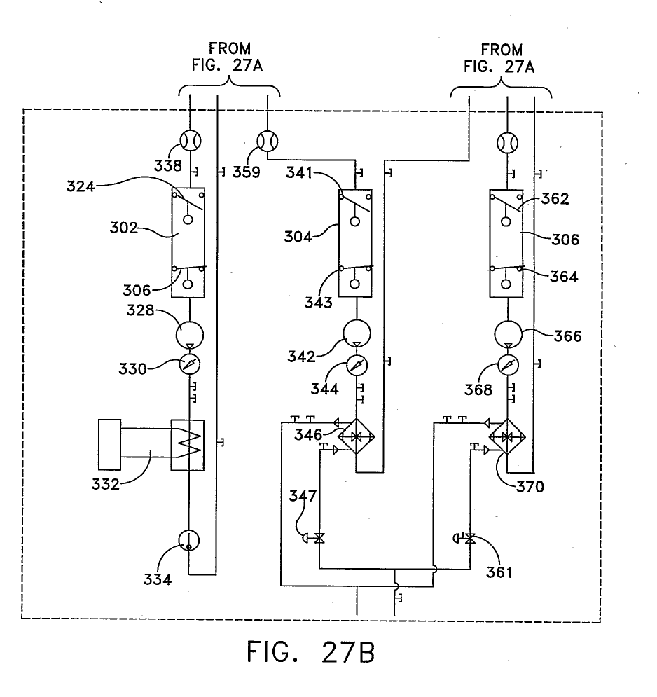

FIGS. 27a and 27b are schematic drawings showing system fluid flow for an embodiment of a 30 kW system. The microwave module includes three inlets (101, 103, 105): saline, CIP and product, controlled by three valves 102, 104, 106. Product flows into the fluid processing unit from one of the inlets through a valve such as a solenoid and into the flow path of the fluid processing module. A bubble sensor 107 can be used to check for bubbles and the motorized pump 112 drives the product flow through a flow meter 111 and pressure sensor 113 and RTD 161. Product flows into the pre-heat, heat exchanger 114 and then past an RTD 162 and into the cartridge 164 where it is exposed to microwave heating. Upon exiting the cartridge 164 product passes an RTD 163 or IR temperature sensor prior to entering a first intercooler 20/40 and a secondary cooler—block plate heat exchanger 160. Product exits the cooling heat exchange section, passes an RTD 166 and a back pressure valve 168 used to avoid fluid boiling before exiting the system through outlet valves 170, 172 to either product or waste containers.

Instrument air supply 450 provides plant air to operate diverting valves 102, 104, 106, 170, 172 and supply pressure to the portion of the wave guide above a pressure window 146 thereby providing low psi pressurization used as a sensor to avoid microwave leakage. Air pressure is also supplied by the instrument air supply to the back pressure valve 168. Electro-pneumatic valves controlled by a PLC, switches air supply to diverting valves 102, 104, 106, 170, 172. Pressure switch 452 insures sufficient pressure to operate diverting valves (also referred to as inlet and outlet valves) properly. Two pressure reducing valves 454 reduce the air pressure supplied to the waveguide, for cartridge sensing, to inches of water. On/off valve 456 allows air flow to waveguide only when the cartridge is in place. Pressure switch 458 provides sensing of sufficient back pressure when cartridge is in proper position.

The hot water section of the utility module provides hot water, from a reservoir 302 with high 324 and low 326 level switches through a pump 328 and pressure gage 330 and into a heater 332. After exiting the heater 332 hot water flows by a first RTD 334 and a second RTD 336 into the preheat heat exchange block 114. Placement of second RTD 336 within fluid processing unit allows adjustment for temperature variation if hot water section is remote at a distance at which temperature drop may occur before entering preheat heat exchange block 114. After flow through the preheat exchange block 114 hot water circulates back to the utility module through a flow meter 338 and into the reservoir 302.

Two cooling loop examples are diagrammed. In this example, the primary loop is diagramed as a parallel flow cold water loop. The primary cold water loop can also be a serial flow loop. The cold water loop can provide cold water or other cold liquid from a reservoir 304 with high 341 and low 343 fluid indicators, through a pump 342 pressure sensor 344 and heat exchanger 346 where the liquid is cooled by an external cold liquid source through valve 347. In this example, fluid flows into the fluid processing unit by an RTD 348 and into the cooling loop chamber 350 surrounding the applicator 122, cooling loop chamber 352 surrounding the auto-tuner, cooling loop chamber 354 surrounding magnetron head, and cooling loop chamber 358 surrounding dummy load before circulating through a pressure sensor 359 and back into the reservoir 304. The cooling loop chamber 356 surrounding microwave control and power supply can be part of the same cooling loop or can be fed with coolant, for example chilled water, from an external source.

In this example, the secondary cooling loop provides cold liquid from a reservoir 306 with high 362 and low 364 fluid indicators through a pump 366 and pressure sensor 368 into a heat exchanger 370 for cooling by cold liquid from an external source through valve 361. Cooled liquid can then flows by an RTD 372 and into the primary 20/40 (for example intercooler) and secondary 160 (for example coolant heat exchangers) before circulating back to the reservoir after passing a flow meter 374.

Also shown in this example is a cabinet cooler 380 with fan 382 to blow cold air. The air can be cooled using a heat exchanger fed by cold water from reservoir 304.

Within the system flow path are temperature test wells and pressure test connections to monitor system parameters and diagnose system problems.

1. A method for selectively reducing, inactivating or destroying an agent in a heat-sensitive material, the method comprising:
a) heating the heat-sensitive material at a rate of at least 50 degrees C. per second to a pre-selected temperature of at least 60 degrees C. to provide a heated liquid material;
b) cooling the heated liquid material at a rate of greater than 100 degrees C. per second;
c) circulating the heat-sensitive material, at a flow rate of greater than 100 liters per hour, during the heating and the cooling;
wherein the reducing or inactivating of the agent occurs while maintaining desirable properties of the heat-sensitive material.

2. The method of claim 1 wherein the heating further comprises exposing the heat-sensitive material to microwave energy, the microwave energy comprising a frequency of at least 1000 MHz.

3. The method of claim 1 wherein the heating further comprises exposing the heat-sensitive material to microwave energy from a microwave generator, the microwave energy comprising a frequency of at least 1000 MHz and the microwave generator has a power capacity of at least 10 kW.

4. The method of claim 1 wherein cooling comprises passing the heated liquid material through a plurality of heat exchangers, said plurality of heat exchangers comprising a tube in shell heat exchanger and a plate heat exchanger and wherein said rate comprises a cooling rate of between 200 and 600 degrees C. per second.

5. A method for reducing, inactivating or destroying an agent in a heat-sensitive material, the method comprising:
a) circulating the heat-sensitive material, at a flow rate of greater than 100 liters per hour,
b) heating the heat-sensitive material through the employment of a microwave generator producing microwave energy having a frequency of at least 1000 MHz and a power capacity of at least 10 kW, for a pre-selected time sufficient to heat the heat-sensitive material at a rate of at least 50 degrees C. per second to a pre-selected temperature of at least 60 degrees C., to provide a heated liquid material; and
c) cooling the heat-sensitive material at a cooling rate of at least 100 degrees C. per second,
wherein the reducing or inactivating of the agent occurs while maintaining desirable properties of the heat-sensitive material.

6-7. (canceled)

8. The method of claim 5 wherein the heating comprises a heating rate of between about 100 degrees per second to about 450 degrees C. per second.

9-10. (canceled)

11. The method of claim 5 wherein the microwave generator produces microwaves having a frequency of 2450+/−50 MHz.

12-14. (canceled)

15. The method of claim 5 wherein the circulating further comprises a flow rate in the range of 100 liters per hour to 500 liters per hour.

16. The method of claim 5 wherein the cooling comprises flowing the heat-sensitive material through a heat exchanger.

17. The method of claim 5 wherein the cooling comprises flowing the heat-sensitive material through a tube and shell heat exchanger.

18. The method of claim 5 wherein the cooling comprises flowing the heat-sensitive fluid through a plurality of heat exchangers.

19. The method of claim 18 wherein one of the plurality of heat exchangers comprises a tube and shell heat exchanger and another of the plurality of heat exchangers comprises a plate heat exchanger.

20. The method of claim 18 further comprising flowing the heat-sensitive material first through a tube and shell heat exchanger and second through a plate heat exchanger.

21. The method of claim 17 wherein the tube and shell heat exchanger comprises a jacketed tube and shell, the jacket comprising a secondary coolant chamber surrounding a primary coolant chamber.

22. The method of 21 wherein the primary and secondary chambers are flowably connected and wherein the coolant in the secondary chamber is received from the primary chamber.

23-32. (canceled)

33. The method of claim 5 wherein the circulating further comprises pumping the heat-sensitive material through tubing of internal diameter of not more than 1 inch.

34. The method of claim 5 wherein the circulating further comprises pumping the heat-sensitive material through tubing of internal diameter of between one-tenth of an inch to one-half an inch.

35-41. (canceled)

42. The method of claim 5 wherein the agent comprises a virus.

43. The method of claim 5 wherein the agent comprises a microorganism.

44. The method of claim 5 wherein the heat-sensitive material comprises a protein and wherein maintaining the desirable properties of the heat-sensitive material comprises reducing the activity of the protein by not more than 90%.

45. The method of claim 5 wherein the heat-sensitive material comprises a protein and wherein maintaining the desirable properties of the heat-sensitive material comprises reducing the activity of the protein by not more than 75%.

46. The method of claim 5 wherein the heat-sensitive material comprises a protein and wherein maintaining the desirable properties of the heat-sensitive material comprises reducing the activity of the protein by not more than 50%.

47-50. (canceled)

51. A system for reducing, inactivating or destroying an agent in a heat-sensitive fluid material, the system comprising:
a) a source of microwave energy producing microwaves with a frequency of greater than 1000 MHz said source of microwave energy comprising a power supply of greater than 10 kW;
b) a flow path for providing a flow stream of the heat-sensitive fluid material, the flow stream having a flow rate of greater than 100 L/hr;
c) a waveguide in microwave contact with the source of microwave energy, the waveguide adapted to receive the flow path for the flow stream within the waveguide;
d) a cooler adapted to receive the flow path for the flow stream after the flow stream exits the waveguide and capable of cooling the flow stream;
e) a control module for controlling components of the system, wherein the control module controls the flow of heat sensitive material through the flow path and controls the application of microwave energy to the heat sensitive material.

52. The system of claim 51, wherein the power supply is contained within a power supply module located remotely from the control module and controlled by the control module.

53. (canceled)

54. The system of claim 51, further comprising a utility module, the utility module located remotely from the control module and controlled by the control module and comprising a source of cooling fluid for the system.

55. (canceled)

56. The system of claim 54 further comprising a heat exchanger for heating the heat-sensitive material in the flow stream prior to the flow stream entering the waveguide and wherein the utility module comprises a source of heating fluid for the heat exchanger.

57. (canceled)

58. The system of claim 51, wherein the cooler comprises a plurality of heat exchangers.

59-60. (canceled)

61. The system of claim 58 wherein one of said heat exchangers comprises a tube and shell heat exchanger and wherein said tube and shell heat exchanger comprises a flow path surrounded by a chamber containing cooling fluid.

62. The system of claim 58 wherein one of the heat exchangers comprises a jacketed tube and shell heat exchanger, the jacket comprising a secondary chamber containing cooling fluid surrounding a primary chamber containing cooling fluid.

63. (canceled)

64. The system of claim 58 wherein one of the plurality of heat exchangers comprises a plate heat exchanger and another comprises a jacketed tube and shell heat exchanger.

65. The system of claim 51 wherein the cooler comprises a plurality of heat exchangers and wherein a first heat exchanger cools at a rate of at least 100 degrees C. per second and a second heat exchanger cools at a rate of not greater than 100 degrees C. per second.

66. The system of claim 51 wherein the heat-sensitive material is heated at a rate of at least 100 degrees C. per second.

67. The system of claim 51 wherein the microwave generator produces microwave energy having a frequency of 2450 MHz+/−50 MHz.

68. The system of claim 67 wherein the microwave generator has a power capacity of between about 10 kW and about 30 kW.

69-74. (canceled)

75. The system of claim 51, wherein the flow path for the flow stream within the waveguide is secured to a removable plate.

76.-96. (canceled)
